(19) United States
(12) Patent Application Publication
(10) Pub. No.: US 2015/0276747 A1
(43) Pub. Date: Oct. 1, 2015

(54) BIOMARKERS FOR OVARIAN CANCER

(71) Applicant: Georgia Regents Research Institute, Inc., Augusta, GA (US)

(72) Inventors: Jin-Xiong She, Martinez, GA (US); Ashok Sharma, Augusta, GA (US)

(21) Appl. No.: 14/560,292

(22) Filed: Dec. 4, 2014

Related U.S. Application Data
(63) Continuation of application No. PCT/US2013/045164, filed on Jun. 11, 2013.
(60) Provisional application No. 61/666,572, filed on Jun. 29, 2012.
(60) Provisional application No. 61/658,123, filed on Jun. 11, 2012.

(51) Int. Cl. G01N 33/574

(52) U.S. Cl. CPC G01N 33/57449; G01N 2570/00; G01N 2333/4703; G01N 2333/70578; G01N 2333/523; G01N 2333/7155; G01N 2333/96494; G01N 2333/705; G01N 2333/522; G01N 2333/70503

(57) ABSTRACT

Biomarkers are provided that are useful for the detection or diagnosis of ovarian cancer. The biomarkers are also useful for determining whether the ovarian cancer is active, is in remission, or is recurring. Preferred biomarkers for detecting or diagnosing ovarian are provided in Table 1. Exemplary combinations of these biomarkers are described in FIGS. 2-6.

### CROSS-REFERENCE TO RELATED APPLICATION

This application is a continuation of International Application No. PCT/US2013/045164 filed under the Patent Cooperation Treaty on Jun. 11, 2013, which claims benefit of and priority to U.S. Provisional Patent Application No. 61/666,572 filed Jun. 29, 2012 and U.S. Provisional Patent Application No. 61/658,123 filed Jun. 11, 2012, all of which are incorporated herein by reference in their entirety.

### FIELD OF THE INVENTION

The invention is generally related to methods of diagnosing and treating ovarian cancer.

### BACKGROUND OF THE INVENTION

Ovarian cancer (OC) is the fifth-leading cause of cancer death among woman in the United States, accounting for approximately 3% of all new cancer patients (1). Worldwide, this disease is the sixth most common cancer in women, causing 140,200 deaths in 2010 (2). Unfortunately, most patients (−70%) are diagnosed with advanced stages of the disease with poor prognosis. Although advances in chemotherapy and improved understanding of genetic risk factor and molecular pathogenesis have provided new treatment possibilities, the 5-year survival rates of late stages are still less than 20% (3). However, the rates of long-term survival (>10 years) in patients diagnosed with early-stage (stage I or II) are 80-95% (4). The lack of successful treatment strategies led to seek novel approaches to detect this disease in early stage and treat this disease effectively in the advanced stage. Recently, there has been a surge of interest in exploring the genome and proteome for biomarkers that may aid in early detection, diagnosis and monitoring of therapeutic outcome and recurrence Previous biomarker research has mostly focused on the discovery and validation of diagnostic biomarkers, especially those that can detect OC at an early stage. The glycoprotein CA125 is the most widely used biomarker for ovarian cancer. It is elevated in approximately 80% of patients with advanced cancer; however, despite its high sensitivity, it lacks specificity and, therefore, has limited positive predictive value (PPV) for population screening, especially for early stage cancer. Extensive search for better biomarkers has been carried out in the last few years and has led to the discovery of a large number of potentially new OC biomarkers including the recently FDA-approved human epididymis protein 4 (HE4) (5, 6). These new biomarkers individually do not perform better than CA125 but biomarker panels with or without CA125 generally perform better than CA125 or other individual biomarkers (7-11). Although the currently available biomarkers do not yet have sufficient PPV suitable for population screening (12), the field of diagnostic biomarkers is a very active and rapidly advancing area of research in ovarian cancer (13).

Biomarkers that allow accurate assessment of therapeutic outcome may significantly improve patient care. After the initial cytoreductive surgery and combination chemotherapy, the majority of OC patients are believed to achieve a complete clinical remission (14). In the remission stage, CA125 is routinely monitored during the followup, and it is widely used as a biomarker for remission. Although CA125 is clearly reduced and returned to levels observed in controls, CA125 levels may not be reliable indicators of the presence of residual cancer cells. After therapy, the patients may have completely remitted or the tumor cell number and size become very small so that the residual tumor cannot be detected by tumor antigens such as CA125. However, as the tumor cells are still present within such patients in subclinical status, the immune system of the patients may be responding to the tumor cells. Therefore, inflammatory molecules may be abnormal in patients with subclinical phenotypes (15-17).

It is an object of the invention to provide compositions and methods for predicting therapeutic outcomes of treatments for ovarian cancer.

It is another object of the invention to provide compositions and methods for the early stage detection of ovarian cancer.

It is still another object of the invention to provide compositions and methods for distinguishing patients in remission from ovarian cancer from healthy subjects or from patients having active ovarian cancer.

### SUMMARY OF THE INVENTION

Biomarkers are provided that are useful for the detection or diagnosis of ovarian cancer. The biomarkers are also useful for determining whether the ovarian cancer is active, is in remission, or is recurring. Preferred biomarkers for detecting or diagnosing ovarian are provided in Table 1. Exemplary combinations of these biomarkers are described in FIGS. 2A-6AC.

One embodiment provides a method for assessing therapeutic outcome of a treatment for ovarian cancer by determining the amount of one or more proteins in a blood sample from a subject in ovarian cancer remission, wherein the one or more proteins are selected from the group consisting of sICAM, sVCAM1, sTNFR-II, sgp130, MMP2, and combinations thereof, and wherein elevated serum amounts of the one or more proteins relative to a control indicates that the subject has poor overall survival relative to subjects in remission for ovarian cancer having lower serum amounts of the one or more serum proteins. Typically groups of 3 to 5 of these markers are assayed. Other biomarkers for ovarian cancer can also be assayed including, for example, CA125.

Methods for treating ovarian cancer include administering to a subject in need thereof one or more chemotherapeutic agents in an amount or for a duration effective to reduce serum levels of one or more proteins selected from the group consisting of sICAM, sVCAM1, sTNFR-II, sgp130, MMP2, and combinations thereof.

Methods for selecting a drug for the treatment of ovarian cancer include administering the drug to a non-human animal model of ovarian cancer, determining the amount of one or more proteins in a blood sample from the non-human animal model, wherein the one or more proteins are selected from the group consisting of sICAM, sVCAM1, sTNFR-II, sgp130, MMP2, and combinations thereof, and selecting the drug that reduces the amounts of the one or more proteins.

Methods for determining the effectiveness of a treatment for ovarian cancer include administering the treatment to a patient in need thereof and measuring the patient's serum levels of one or more proteins selected from the group consisting of sICAM, sVCAM1, sTNFR-II, sgp130, MMP2, and combinations thereof, wherein decreased levels of the one or more proteins relative to a control indicates that the treatment is effective.

Methods for determining the effectiveness of a cancer treatment include determining serum levels of biomarkers of inflammation in a subject before and after treatment wherein a decrease in serum levels of biomarkers of inflammation after treatment indicates that the treatment is effective.

Methods for detecting or diagnosing ovarian cancer in a subject include determining the serum levels of one or more proteins selected from the group consisting of PDGF-AA/BB, PDGF-AA, CRP, sFas, sTNFR-II, SAA, sIL-6R, MMP-1, and sCD40L and combinations thereof, wherein elevated serum levels of one or more of CRP, sFas, sTNFR-II, SAA, sIL-6R, MMP-1, and sCD40L and reduced serum levels of one or more of PDGF-AA/BB, PDGF-AA and combinations thereof are indicative of ovarian cancer.

Methods for identifying subjects having active ovarian cancer include determining the serum levels of CRP in a subject after the subject has been treated for the ovarian cancer, wherein elevated serum levels of CRP is indicative of active ovarian cancer in the subject.

Methods for determining survivability of a patient in ovarian cancer remission include assaying the serum levels of one or more proteins selected from the group consisting of sICAM1, sTNFR-II, RANTES, sgp130, CA15-3, MIG, MMP-2, sVCAM-1, TPO, sTNFR-I and MDC and combinations thereof from a blood sample obtained from the patient in ovarian cancer remission, wherein elevated levels of the one or more proteins and/or the reduced level of MDC relative to a control is indicated of reduced survivability relative to patients in ovarian cancer remission having reduced serum levels of the one or more proteins.

### BRIEF DESCRIPTION OF THE DRAWINGS

FIGS. 1A-1J show boxplots representing the serum protein levels in patient subgroups and healthy controls. PD: Post Diagnosis, RC: Recurrence, RM: Remission, HC: Healthy Controls.

FIGS. 2A-2T show the ROC curves for the top molecules that can distinguish cancer patients (post diagnosis and recurrence) from healthy controls. Single proteins (FIGS. 2A-2J) and multi-marker models (FIGS. 2K-2T) were used for the classification analyses. For multi-marker models, linear discriminate analysis was performed using combinations of 3 proteins. The diagnostic performance of each model was evaluated using leave one out cross validation method. The utility of serum proteins as ovarian cancer biomarkers was evaluated using the area-under-curve (AUC) of the ROC curves for different models.

FIGS. 3A-3T show the ROC curves for the top molecules that distinguish samples at remission from samples with active cancer (FIGS. 3A-3H) or healthy controls (FIGS. 3I-3T). Results were shown for single proteins (FIGS. 3A-3D and 3I-3N) and multi-marker models (FIGS. 3E-3H and 3O-3T).

FIGS. 4A and 4B-4E show the survival analyses of ovarian cancer patients. Kaplan-Meier analysis was used to investigate the relationship of individual protein levels on overall survival in three different phenotypic groups (PD, RC, and RM). In FIG. 4A, the subjects were assigned to the low or high expression groups based on the protein expression for each protein. FIGS. 4B-4E show the representative survival curves of the samples from the PD stage using single proteins and a combination of 4 protein model.

FIGS. 5A-5K show the survival analyses of the samples from the RM stage. The survival curves for the top five molecules that can distinguish patient subset with poor overall survival from patients with better survival. The prognostic value of multivariate models (combinations of 4 or 5 proteins) was determined by clustering the patients into two groups based on the expression levels of protein panels and survival differences were then determined between these two clusters using Kaplan-Meier analyses. The heatmap of protein expression in the samples from the RM stage (FIG. 5L). The patients with poor survival have higher expression levels for the five proteins.

FIGS. 6A-6AC show additional survival analyses of ovarian cancer patients.

### DETAILED DESCRIPTION OF THE INVENTION

### I. Definitions

In describing and claiming the disclosed subject matter, the following terminology will be used in accordance with the definitions set forth below.

As used herein, “treat” means to prevent, reduce, decrease, or ameliorate one or more symptoms, or characteristics of cancer, in particular ovarian cancer, to halt the progression of one or more symptoms, or characteristics of ovarian cancer.

The terms “individual,” “subject,” and “patient” are used interchangeably herein, and refer to a mammal, including, but not limited to, rodents, simians, and humans.

The terms “reduce”, “inhibit”, “alleviate” and “decrease” are used relative to a control. One of skill in the art would readily identify the appropriate control to use for each experiment. For example a decreased response in a subject or cell treated with a compound is compared to a response in subject or cell that is not treated with the compound.

The term “remission” in relation to cancer refers to a decrease in or disappearance of signs and symptoms of cancer. In partial remission, some, but not all, signs and symptoms of cancer have disappeared. In complete remission, all signs and symptoms of cancer have disappeared or are undetectable.

The term “biological sample”, “sample”, and “test sample” are used interchangeably herein to refer to any material, biological fluid, tissue, or cell obtained or otherwise derived from an individual. This includes blood (including whole blood, leukocytes, peripheral blood mononuclear cells, buffy coat, plasma, and serum), sputum, tears, mucus, nasal washes, nasal aspirate, breath, urine, semen, saliva, meningeal fluid, amniotic fluid, glandular fluid, lymph fluid, nipple aspirate, bronchial aspirate, synovial fluid, joint aspirate, cells, a cellular extract, and cerebrospinal fluid. This also includes experimentally separated fractions of all of the preceding. For example, a blood sample can be fractionated into serum or into fractions containing particular types of blood cells, such as red blood cells or white blood cells (leukocytes). If desired, a sample can be a combination of samples from an individual, such as a combination of a tissue and fluid sample. The term “biological sample” also includes materials containing homogenized solid material, such as from a stool sample, a tissue sample, or a tissue biopsy, for example. The term “biological sample” also includes materials derived from a tissue culture or a cell culture. The biological sample can be obtained using conventional techniques including but not limited to phlebotomy, swab (e.g., buccal swab), and a fine needle aspirate biopsy procedure. Exemplary tissues susceptible to fine needle aspiration include ovaries, lymph node, lung, lung washes, BAL (bronchoalveolar lavage), thyroid, breast, and liver. Samples can also be collected, e.g., by micro dissection (e.g., laser capture micro dissection (LCM) or laser micro dissection (LMD)), bladder wash, smear (e.g., a PAP smear), or ductal lavage. A “biological sample” obtained or derived from an individual includes any such sample that has been processed in any suitable manner after being obtained from the individual.

The terms “marker” and “biomarker” are used interchangeably to refer to a target molecule that indicates or is a sign of a normal or abnormal process in an individual or of a disease or other condition in an individual. More specifically, a “marker” or “biomarker” is an anatomic, physiologic, biochemical, or molecular parameter associated with the presence of a specific physiological state or process, whether normal or abnormal, and, if abnormal, whether chronic or acute. Biomarkers are detectable and measurable by a variety of methods including laboratory assays and medical imaging. When a biomarker is a protein, it is also possible to use the expression of the corresponding gene as a surrogate measure of the amount or presence or absence of the corresponding protein biomarker in a biological sample or methylation state of the gene encoding the biomarker or proteins that control expression of the biomarker.

The term “biomarker value”, “value”, “biomarker level”, and “level” are used interchangeably to refer to a measurement that is made using any analytical method for detecting the biomarker in a biological sample and that indicates the presence, absence, absolute amount or concentration, relative amount or concentration, titer, an expression level, a ratio of measured levels, or the like, of, for, or corresponding to the biomarker in the biological sample. The exact nature of the “value” or “level” depends on the specific design and components of the particular analytical method employed to detect the biomarker.

When a biomarker indicates or is a sign of an abnormal process or a disease or other condition in an individual, that biomarker is generally described as being either over-expressed or under-expressed as compared to an expression level or value of the biomarker that indicates or is a sign of a normal process or an absence of a disease or other condition in an individual. “Up-regulation”, “up-regulated”, “over-expression”, “over-expressed”, and any variations thereof are used interchangeably to refer to a value or level of a biomarker in a biological sample that is greater than a value or level (or range of values or levels) of the biomarker that is typically detected in similar biological samples from healthy or normal individuals. The terms may also refer to a value or level of a biomarker in a biological sample that is greater than a value or level (or range of values or levels) of the biomarker that may be detected at a different stage of a particular disease.

“Down-regulation”, “down-regulated”, “under-expression”, “under-expressed”, and any variations thereof are used interchangeably to refer to a value or level of a biomarker in a biological sample that is less than a value or level (or range of values or levels) of the biomarker that is typically detected in similar biological samples from healthy or normal individuals. The terms may also refer to a value or level of a biomarker in a biological sample that is less than a value or level (or range of values or levels) of the biomarker that may be detected at a different stage of a particular disease.

Further, a biomarker that is either over-expressed or under-expressed can also be referred to as being “differentially expressed” or as having a “differential level” or “differential value” as compared to a “normal” expression level or value of the biomarker that indicates or is a sign of a normal process or an absence of a disease or other condition in an individual. Thus, “differential expression” of a biomarker can also be referred to as a variation from a “normal” expression level of the biomarker.

The term “differential gene expression” and “differential expression” are used interchangeably to refer to a gene (or its corresponding protein expression product) whose expression is activated to a higher or lower level in a subject suffering from a specific disease, relative to its expression in a normal or control subject. The terms also include genes (or the corresponding protein expression products) whose expression is activated to a higher or lower level at different stages of the same disease. It is also understood that a differentially expressed gene may be either activated or inhibited at the nucleic acid level or protein level, or may be subject to alternative splicing to result in a different polypeptide product. Such differences may be evidenced by a variety of changes including mRNA levels, surface expression, secretion or other partitioning of a polypeptide. Differential gene expression may include a comparison of expression between two or more genes or their gene products; or a comparison of the ratios of the expression between two or more genes or their gene products; or even a comparison of two differently processed products of the same gene, which differ between normal subjects and subjects suffering from a disease; or between various stages of the same disease. Differential expression includes both quantitative, as well as qualitative, differences in the temporal or cellular expression pattern in a gene or its expression products among, for example, normal and diseased cells, or among cells which have undergone different disease events or disease stages.

As used herein, “individual” refers to a test subject or patient. The individual can be a mammal or a non-mammal. In various embodiments, the individual is a mammal A mammalian individual can be a human or non-human. In various embodiments, the individual is a human. A healthy or normal individual is an individual in which the disease or condition of interest (including, for example, ovarian cancer) is not detectable by conventional diagnostic methods.

“Diagnose”, “diagnosing”, “diagnosis”, and variations thereof refer to the detection, determination, or recognition of a health status or condition of an individual on the basis of one or more signs, symptoms, data, or other information pertaining to that individual. The health status of an individual can be diagnosed as healthy/normal (i.e., a diagnosis of the absence of a disease or condition) or diagnosed as ill/abnormal (i.e., a diagnosis of the presence, or an assessment of the characteristics, of a disease or condition). The terms “diagnose”, “diagnosing”, “diagnosis”, etc., encompass, with respect to a particular disease or condition, the initial detection of the disease; the characterization or classification of the disease; the detection of the progression, remission, or recurrence of the disease; and the detection of disease response after the administration of a treatment or therapy to the individual. The diagnosis of ovarian cancer includes distinguishing individuals, including smokers and nonsmokers, who have cancer from individuals who do not. It further includes distinguishing benign masses from cancerous masses.

“Prognose”, “prognosing”, “prognosis”, and variations thereof refer to the prediction of a future course of a disease or condition in an individual who has the disease or condition (e.g., predicting patient survival), and such terms encompass the evaluation of disease response after the administration of a treatment or therapy to the individual.

“Evaluate”, “evaluating”, “evaluation”, and variations thereof encompass both “diagnose” and “prognose” and also encompass determinations or predictions about the future course of a disease or condition in an individual who does not have the disease as well as determinations or predictions regarding the likelihood that a disease or condition will recur in an individual who apparently has been cured of the disease. The term “evaluate” also encompasses assessing an individual's response to a therapy, such as, for example, predicting whether an individual is likely to respond favorably to a therapeutic agent or is unlikely to respond to a therapeutic agent (or will experience toxic or other undesirable side effects, for example), selecting a therapeutic agent for administration to an individual, or monitoring or determining an individual's response to a therapy that has been administered to the individual. Thus, “evaluating” ovarian cancer can include, for example, any of the following: prognosing the future course of ovarian cancer in an individual; predicting the recurrence of ovarian cancer in an individual who apparently has been cured of ovarian cancer; or determining or predicting an individual's response to a ovarian cancer treatment or selecting an ovarian cancer treatment to administer to an individual based upon a determination of the biomarker values derived from the individual's biological sample.

Any of the following examples may be referred to as either “diagnosing” or “evaluating” ovarian cancer: initially detecting the presence or absence of ovarian cancer; determining a specific stage, type or sub-type, or other classification or characteristic of ovarian cancer; determining whether a mass is a benign lesion or a malignant tumor; or detecting/monitoring ovarian cancer progression (e.g., monitoring ovarian tumor growth or metastatic spread), remission, or recurrence.

As used herein, “additional biomedical information” refers to one or more evaluations of an individual, other than using any of the biomarkers described herein, that are associated with ovarian cancer risk. “Additional biomedical information” includes any of the following: physical descriptors of an individual, physical descriptors of a ovarian mass observed by CT imaging, the height and/or weight of an individual, the gender of an individual, the ethnicity of an individual, smoking history, occupational history, exposure to known carcinogens (e.g., exposure to any of asbestos, radon gas, chemicals, smoke from fires, and air pollution, which can include emissions from stationary or mobile sources such as industrial/factory or auto/marine/aircraft emissions), exposure to second-hand smoke, family history of ovarian cancer (or other cancer), the presence of nodules, size of nodules, location of nodules, morphology of nodules (e.g., as observed through CT imaging, ground glass opacity (GGO), solid, non-solid), edge characteristics of the nodule (e.g., smooth, lobulated, sharp and smooth, spiculated, infiltrating), and the like. Additional biomedical information can be obtained from an individual using routine techniques known in the art, such as from the individual themselves by use of a routine patient questionnaire or health history questionnaire, etc., or from a medical practitioner, etc. Alternately, additional biomedical information can be obtained from routine imaging techniques, including CT imaging (e.g., low-dose CT imaging) and X-ray. Testing of biomarker levels in combination with an evaluation of any additional biomedical information may, for example, improve sensitivity, specificity, and/or AUC for detecting ovarian cancer (or other ovarian cancer-related uses) as compared to biomarker testing alone or evaluating any particular item of additional biomedical information alone (e.g., CT imaging alone).

The term “area under the curve” or “AUC” refers to the area under the curve of a receiver operating characteristic (ROC) curve, both of which are well known in the art. AUC measures are useful for comparing the accuracy of a classifier across the complete data range. Classifiers with a greater AUC have a greater capacity to classify unknowns correctly between two groups of interest (e.g., ovarian cancer samples and normal or control samples). ROC curves are useful for plotting the performance of a particular feature (e.g., any of the biomarkers described herein and/or any item of additional biomedical information) in distinguishing between two populations (e.g., cases having ovarian cancer and controls without ovarian cancer). Typically, the feature data across the entire population (e.g., the cases and controls) are sorted in ascending order based on the value of a single feature. Then, for each value for that feature, the true positive and false positive rates for the data are calculated. The true positive rate is determined by counting the number of cases above the value for that feature and then dividing by the total number of cases. The false positive rate is determined by counting the number of controls above the value for that feature and then dividing by the total number of controls. Although this definition refers to scenarios in which a feature is elevated in cases compared to controls, this definition also applies to scenarios in which a feature is lower in cases compared to the controls (in such a scenario, samples below the value for that feature would be counted). ROC curves can be generated for a single feature as well as for other single outputs, for example, a combination of two or more features can be mathematically combined (e.g., added, subtracted, multiplied, etc.) to provide a single sum value, and this single sum value can be plotted in a ROC curve. Additionally, any combination of multiple features, in which the combination derives a single output value, can be plotted in a ROC curve. These combinations of features may comprise a test. The ROC curve is the plot of the true positive rate (sensitivity) of a test against the false positive rate (1-specificity) of the test.

As used herein, “detecting” or “determining” with respect to a biomarker level or value includes the use of both the instrument required to observe and record a signal corresponding to a biomarker level or value and the material/s required to generate that signal. In various embodiments, the biomarker level or value is detected using any suitable method, including fluorescence, chemiluminescence, surface plasmon resonance, surface acoustic waves, mass spectrometry, infrared spectroscopy, Raman spectroscopy, atomic force microscopy, scanning tunneling microscopy, electrochemical detection methods, nuclear magnetic resonance, quantum dots, and the like.

### II. Biomarkers for Detection, Diagnosis, Prognosis and Predict Therapeutic Outcome of Ovarian Cancer

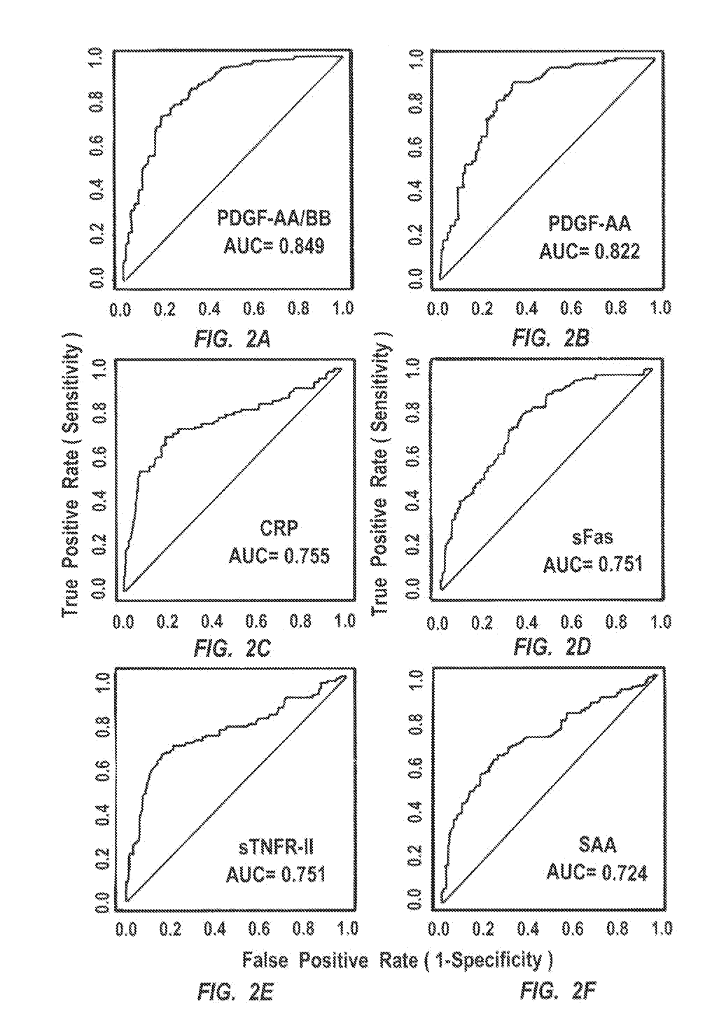

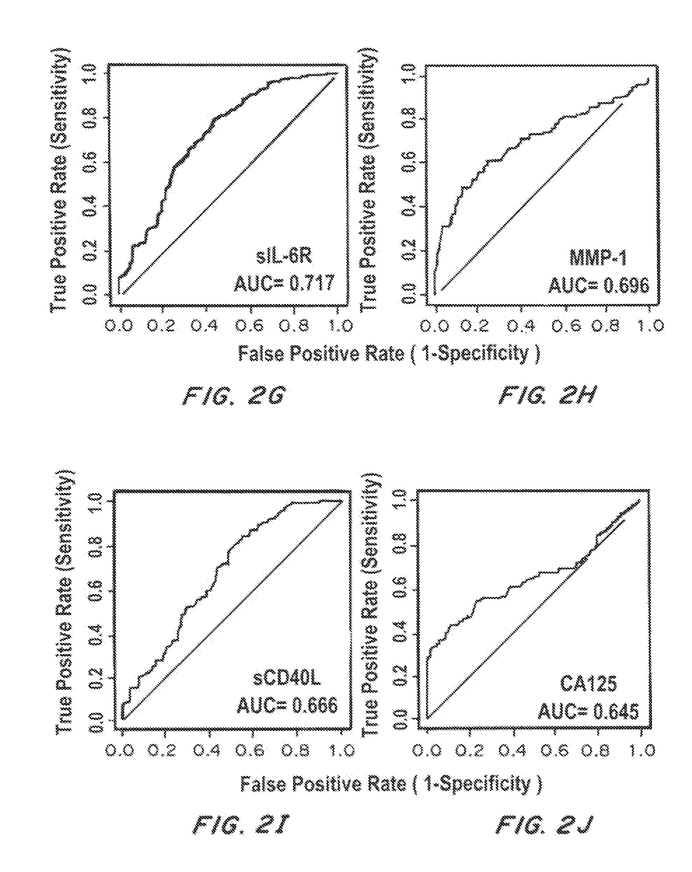

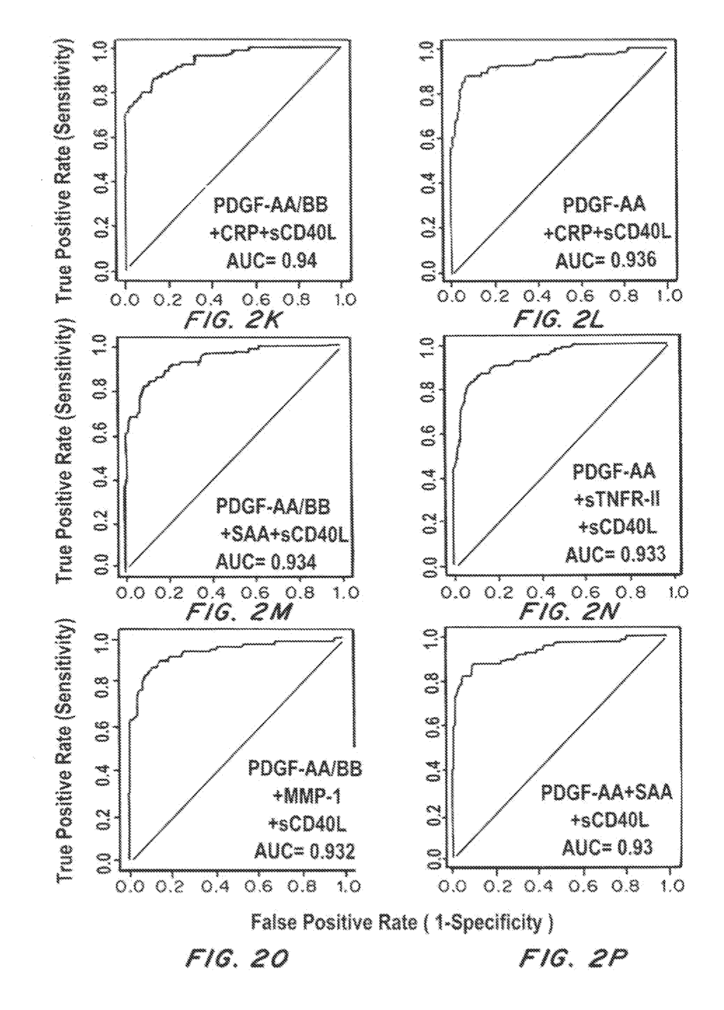

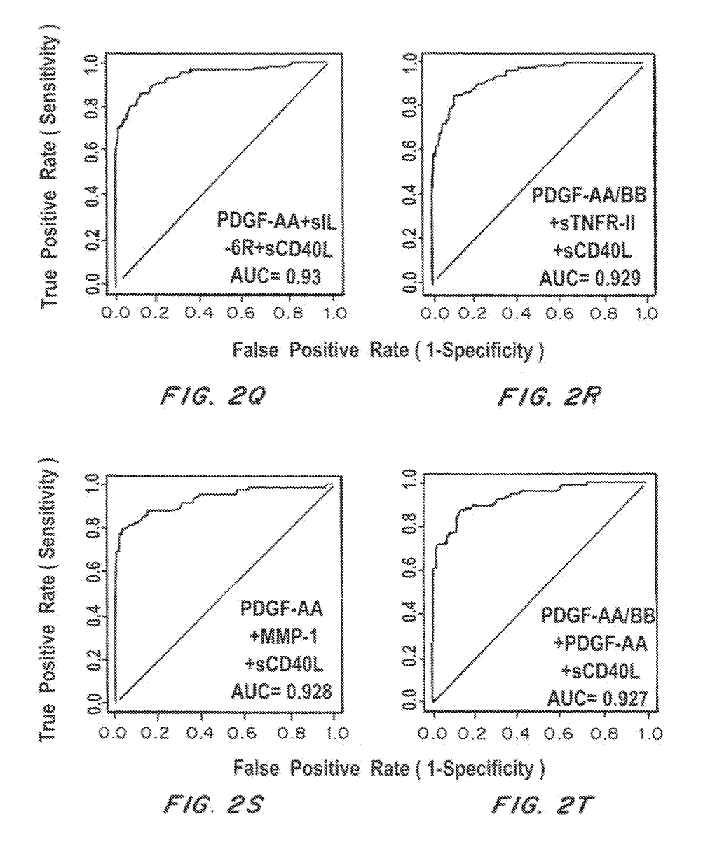

Serum protein profiles have been discovered that can be used to distinguish ovarian cancer patients with active cancer from healthy controls and/or ovarian cancer patients that are at remission, and to predict the therapeutic outcome of ovarian cancer treatments. In general, 28 proteins were identified that show differences between healthy subjects, subjects with active ovarian cancer, and subjects in complete or partial remission for ovarian cancer (Table 1). Although the individual serum proteins can be used as indicators of ovarian cancer, groups of two, three, four, or even five of the identified serum proteins provided better predictability (FIGS. 2A-6AC). Eleven serum proteins at RM stage accurately predict therapeutic outcomes. The eleven serum proteins include sICAM1, sVCAM1, sgp130, MMP2, sTNFR-II, CA15-3, MIG, sVCAM-1, TPO, sTNFR-I and MDC.

A. Twenty-Eight Serum Protein Profiles are Altered in Subject Having Ovarian Cancer

28 proteins have been identified that exhibit altered serum levels in patients having OC compared to HC (Table 1). Ratios of PD/HC, RC/HC and RM/HC shown in Table 1 that are less than 1.0 indicate that the specific protein is present at reduced serum levels relative to healthy controls. Ratios in Table 1 that are greater than 1.0 indicate that the specific protein is present at higher serum levels relative to healthy controls.

One embodiment provides a method for detecting or diagnosing ovarian cancer in a subject by determining the serum levels in a sample obtained from the subject of one or more of the proteins listed in Table 1 including: growth factor AA/BB (PDGF-AA/AB), soluble CD40 ligand (sCD40L), platelet derived growth factor A (PDGF-AA), C-reactive protein (CRP), serum amyloid A (SAA), metalloproteinase-1 (MMP-1), insulin-like growth factor binding protein 2 (IGFBP-2), cancer antigen 125 (CA125), Leptin, soluble tumor necrosis factor II (sTNFR-II), soluble Fas (sFas), soluble interleukin 2 receptor A (sIL-2Ra), CD14, soluble interleukin 6 receptor (sIL-6R), insulin-like growth factor binding protein 6 (IGFBP-6), tissue plasminogen activator inhibitor-1 (tPAI-1), hepatocyte growth factor (HGF), soluble vascular cell adhesion protein 1 (sVCAM-1), soluble E-selectin (sE-selectin), macrophage-derived chemokine (MDC), insulin-like growth factor binding protein 3 (IGFBP-3), and metalloproteinase-2 (MMP-2).

The proteins showing the highest increase is serum levels include CRP and SAA, indicating active inflammation in the patients with active disease (PD and RC) but to a lesser degree at the remission stage (RM). Inflammation in OC is also indicated by the increased levels of soluble receptors such as sTNFR-II and sCD40L.

The most down-regulated proteins are PDGF-AA/BB and PDGF-AA, two related molecules which play an important role in cell proliferation and angiogenesis. Genomic studies suggested that activation of the PDGF pathway plays an important role in OC (35). While the pro-angiogenic and pro-growth function of PDGF would predict higher levels of serum PDGF (36), these two proteins are surprisingly lower in OC patients compared to HC.

B. Biomarkers for Predicting Therapeutic Outcome

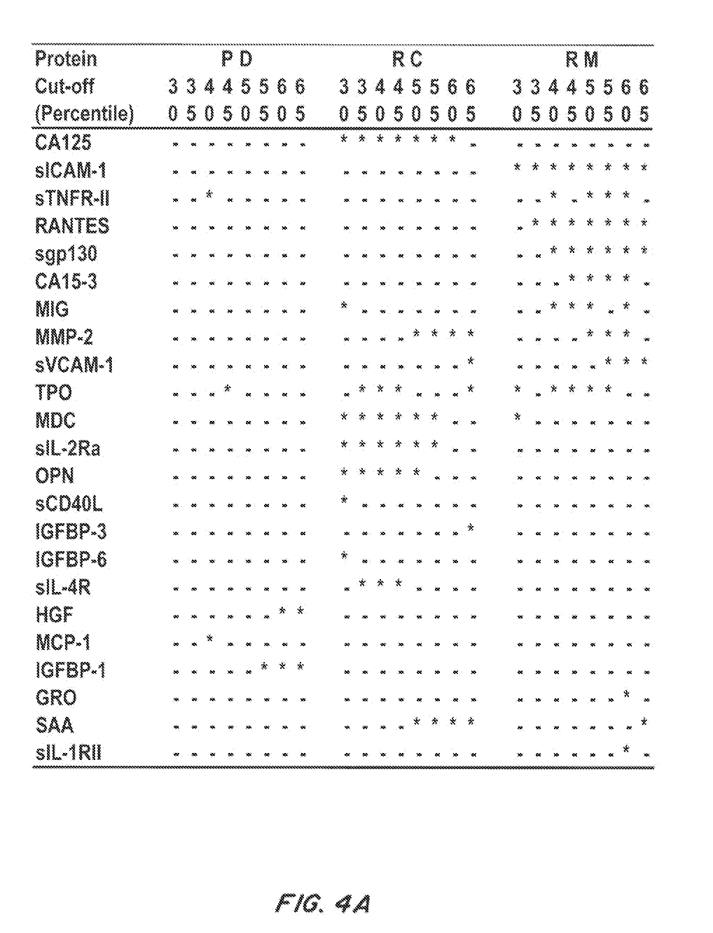

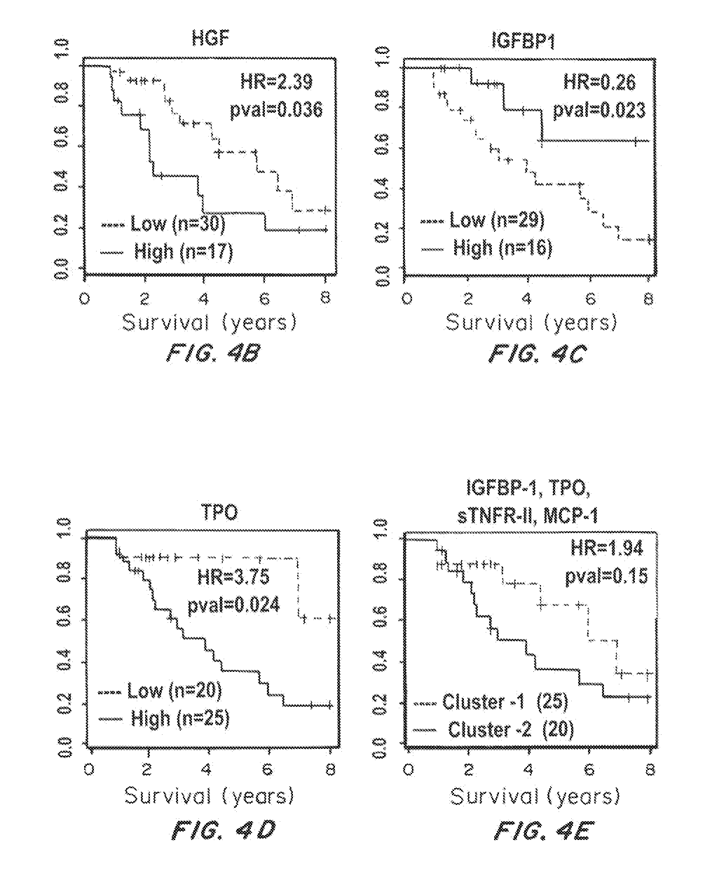

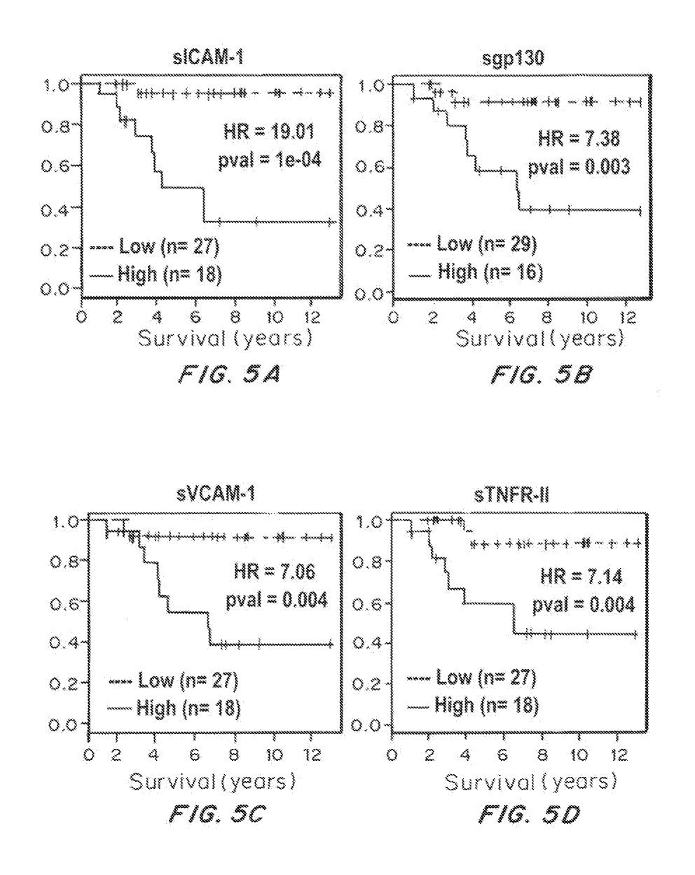

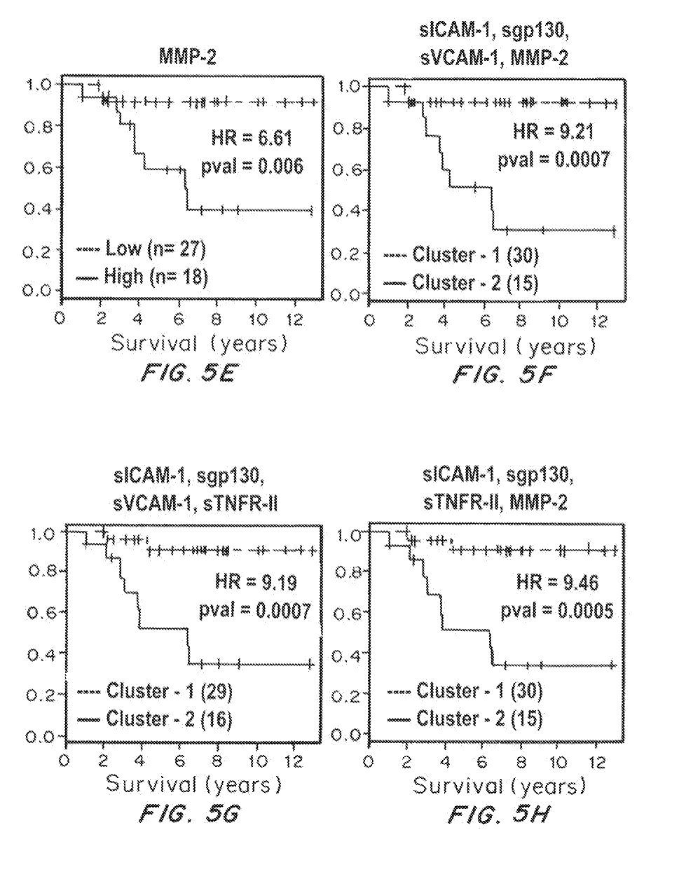

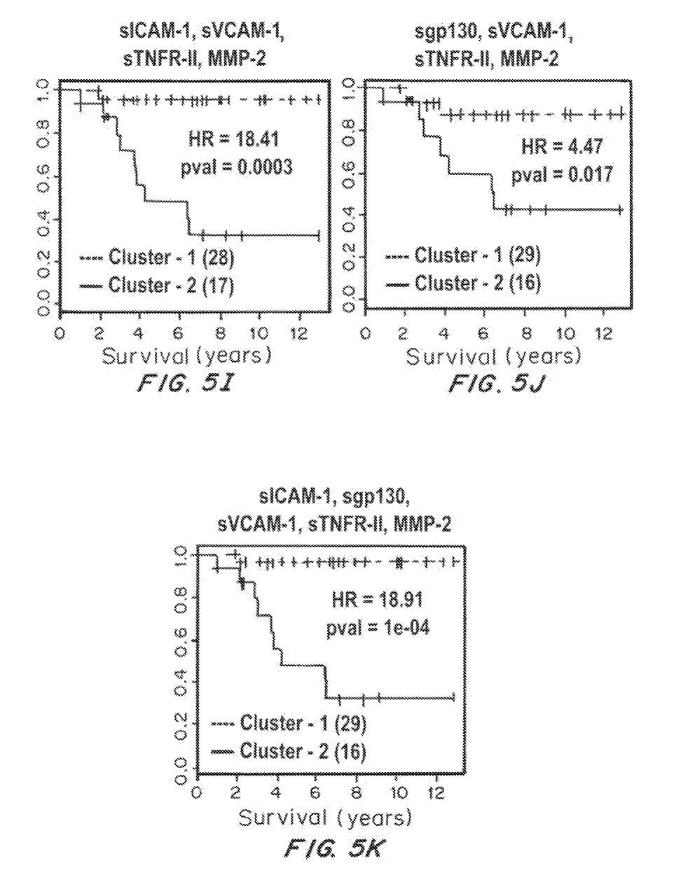

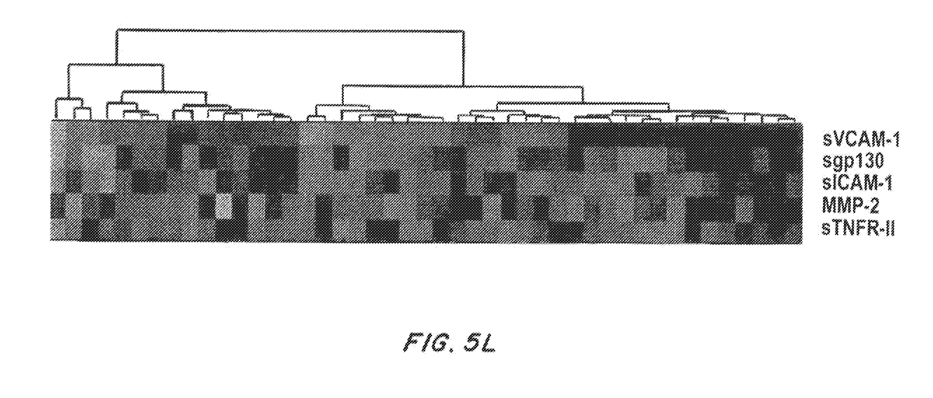

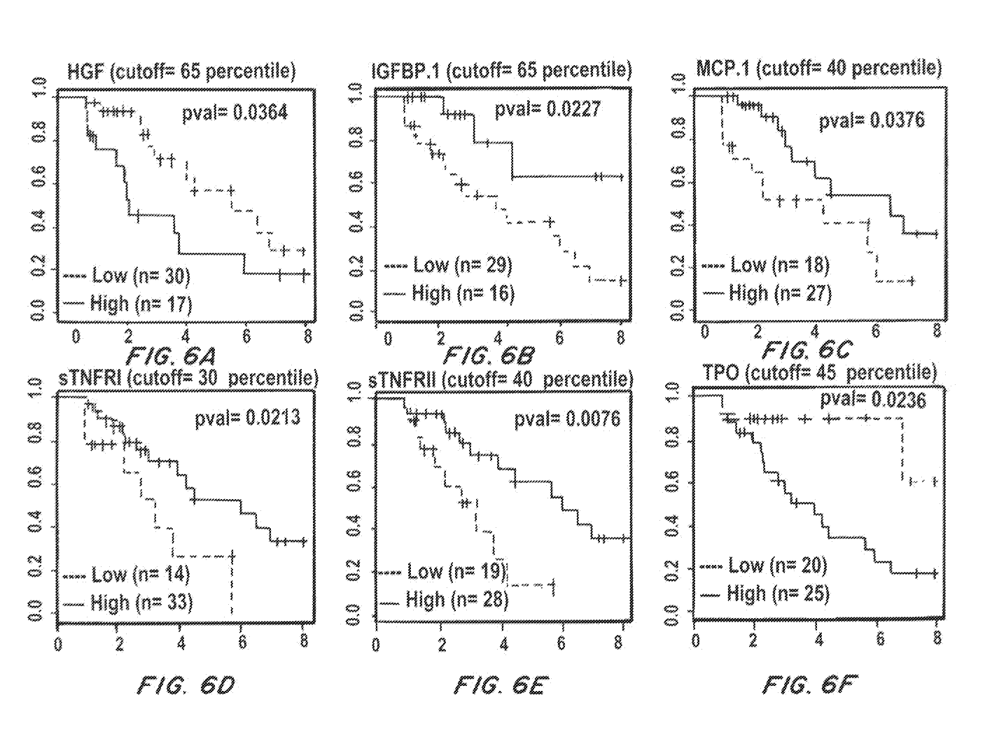

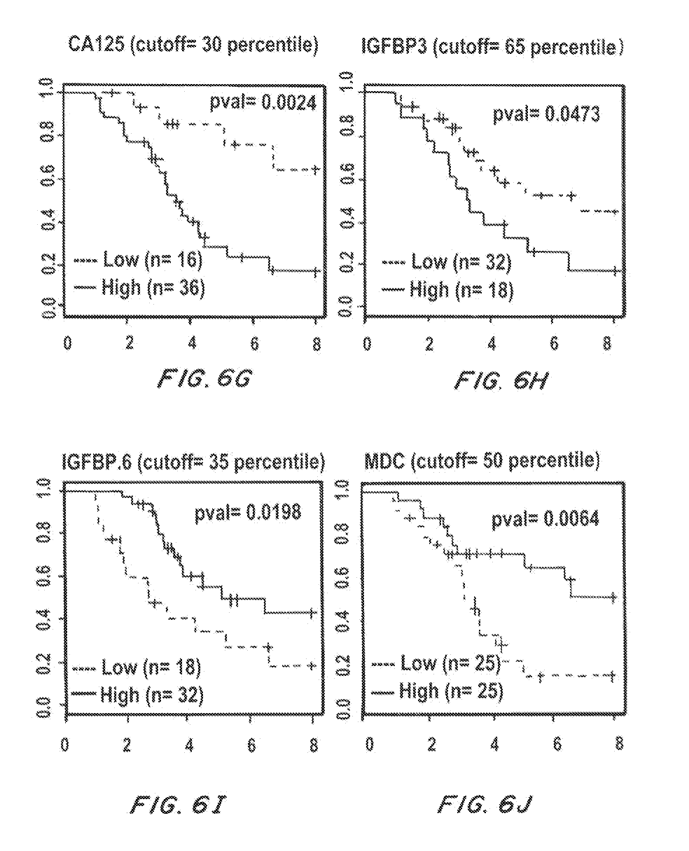

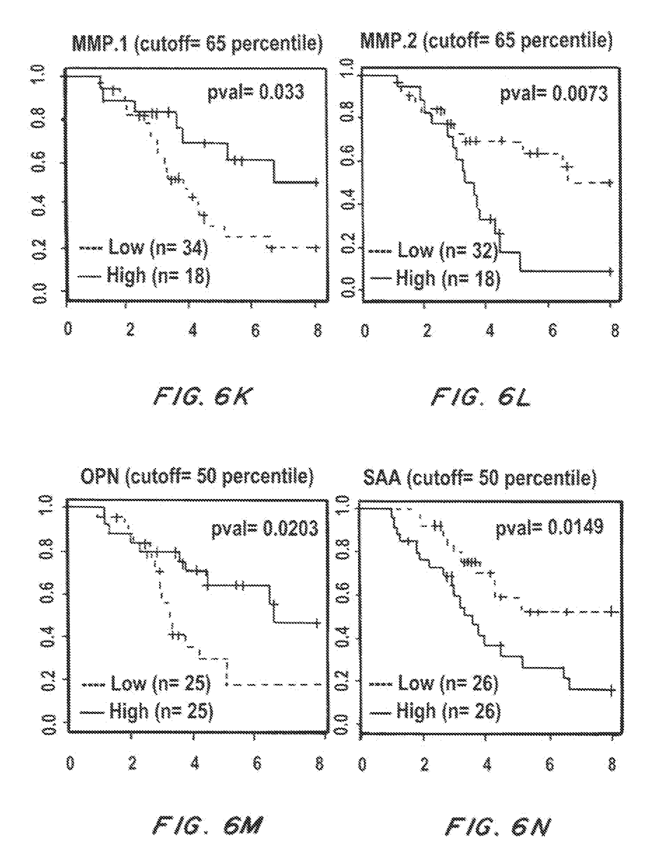

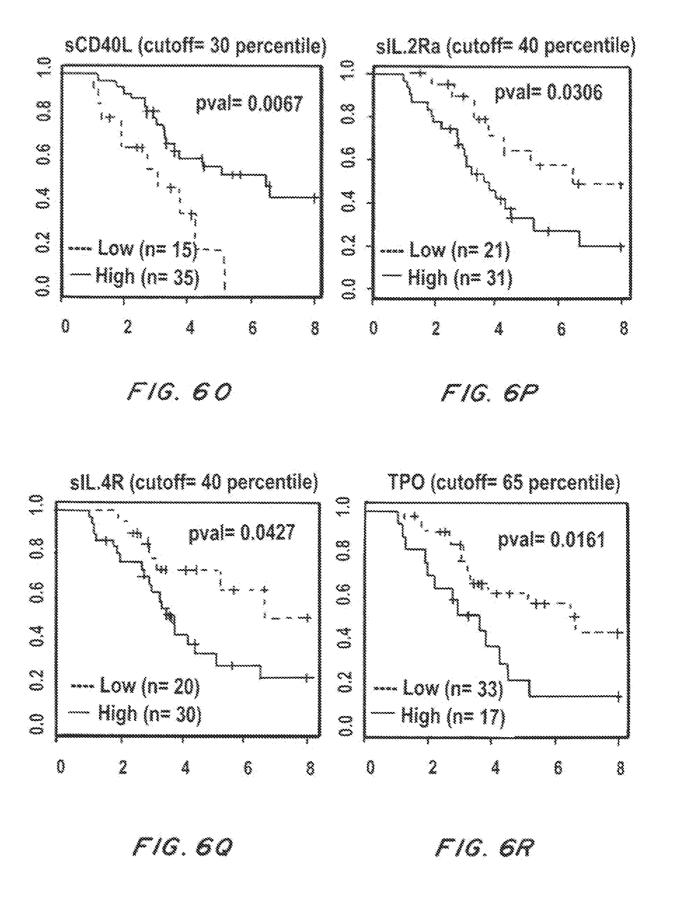

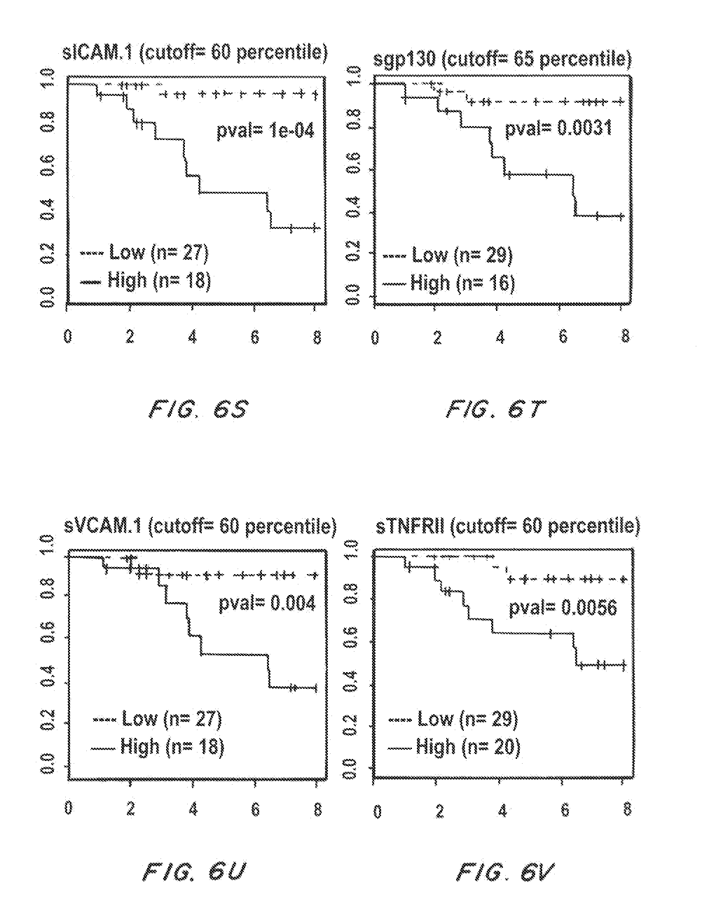

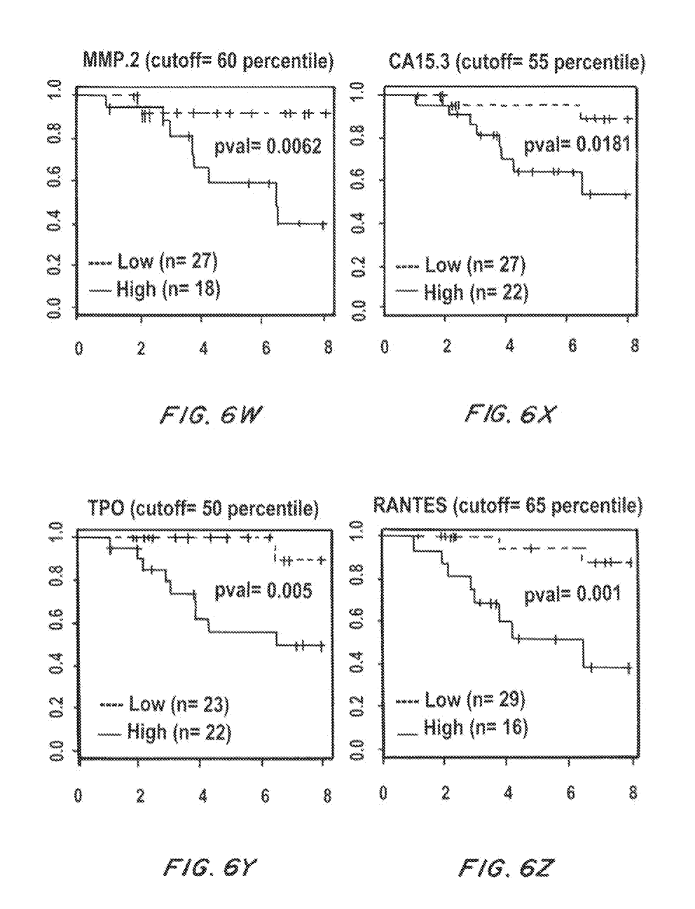

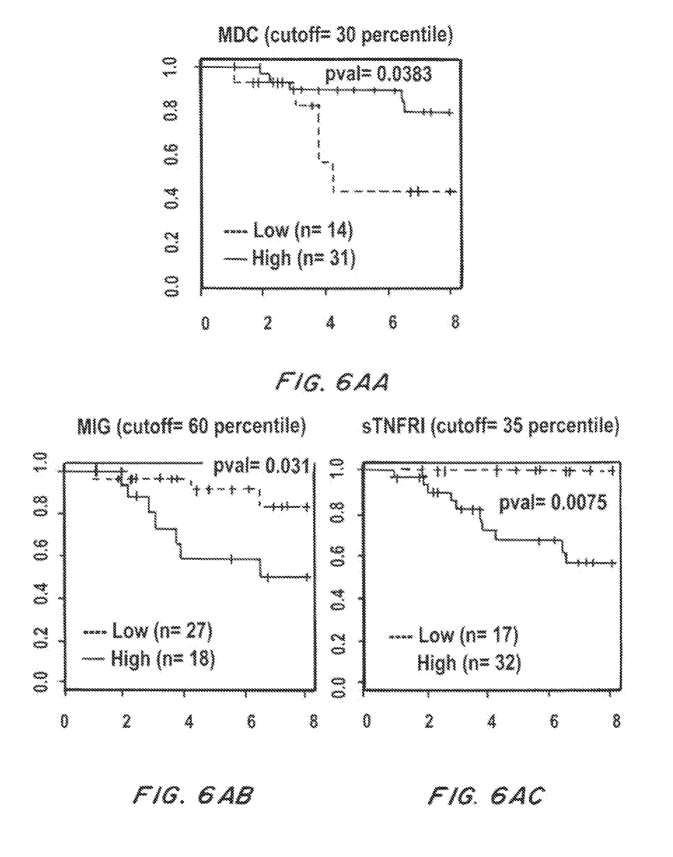

Multiple proteins (sICAM1, sTNFR-II, RANTES, sgp130, CA15-3, MIG, MMP-2, sVCAM-1, TPO, sTNFR-I and MDC) measured at the RM stage can individually predict overall survival of OC patients (FIG. 4A and FIGS. 6A-6AC). Among these proteins, five (sICAM1, sVCAM1, sgp130, MMP2, sTNFR-II) could separate the RM patients into two subgroups with distinct prognosis and sICAM-1 had the best prognostic value (HR=19.01, p=10⁻⁴, FIGS. 5A-5K). The prognostic value of all 5 models using 4 of the 5 proteins and the 5-protein model (FIGS. 5A-5K) was also evaluated. All five 4-protein models have excellent prognostic potential while the 5-protein model has the best performance (p=10⁻⁴ and MR=18.91). In the five-protein model, only one of the 29 patients in Cluster 1 did not survive, while 9 of the 16 patients in cluster 2 died during the follow-up period. Interestingly, the heat-map of protein expression (FIG. 5L) clearly shows that the patients with poor survival have higher expression levels for the five proteins.

### III. Methods of Using the Biomarkers for Ovarian Cancer

A. Diagnosis

One embodiment provides a method in which a biological sample obtained from a subject of interest, preferably a human subject, is assayed to detect the presence of or quantitate the amount (i.e., relative amount) of one or more of the twenty-eight biomarkers for ovarian cancer described herein, for example in Table 1. Exemplary combinations of these markers can also be used as described in FIGS. 2A-6AC.

Methods for diagnosing ovarian cancer in an individual include determining levels or values of one ore more of the disclosed biomarkers in Table 1 present in the circulation of an individual, such as in serum or plasma, using conventional analytical methods. These biomarkers are, for example, differentially expressed in individuals with ovarian cancer as compared to individuals without ovarian cancer. Detection of the differential expression of a biomarker in an individual can be used, for example, to permit the early diagnosis of ovarian cancer, to distinguish between a benign and malignant masses (such as, for example, a nodule observed on a computed tomography (CT) scan), to monitor ovarian cancer recurrence, monitor ovarian cancer remission or for other clinical indications.

Any of the biomarkers described herein may be used in a variety of clinical indications for ovarian cancer, including any of the following: detection of ovarian cancer (such as in a high-risk individual or population); characterizing ovarian cancer (e.g., determining ovarian cancer type, sub-type, or stage); determining whether an ovarian nodule is a benign nodule or a malignant ovarian tumor; determining ovarian cancer prognosis; monitoring ovarian cancer progression or remission; monitoring for ovarian cancer recurrence; monitoring metastasis; treatment selection; monitoring response to a therapeutic agent or other treatment; stratification of individuals for computed tomography (CT) screening (e.g., identifying those individuals at greater risk of ovarian cancer and thereby most likely to benefit from spiral-CT screening, thus increasing the positive predictive value of CT); combining biomarker testing with additional biomedical information, such as family history of ovarian cancer, etc., or with nodule size, morphology, etc. (such as to provide an assay with increased diagnostic performance compared to CT testing or biomarker testing alone); facilitating the diagnosis of an ovarian nodule as malignant or benign; facilitating clinical decision making once an ovarian nodule is observed on CT (e.g., ordering repeat CT scans if the nodule is deemed to be low risk, such as if a biomarker-based test is negative, with or without categorization of nodule size, or considering biopsy if the nodule is deemed medium to high risk, such as if a biomarker-based test is positive, with or without categorization of nodule size); and facilitating decisions regarding clinical follow-up (e.g., whether to implement repeat CT scans, fine needle biopsy, after observing a non-calcified nodule on CT). Biomarker testing may improve positive predictive value (PPV) over CT screening alone. In addition to their utilities in conjunction with CT screening, the biomarkers described herein can also be used in conjunction with any other imaging modalities used for ovarian cancer, such as magnetic resonance imaging (MRI) scans and ultrasound studies. Furthermore, the described biomarkers may also be useful in permitting certain of these uses before indications of ovarian cancer are detected by imaging modalities or other clinical correlates, or before symptoms appear.

As an example of the manner in which any of the biomarkers described herein can be used to diagnose ovarian cancer, differential expression of one or more of the described biomarkers in an individual who is not known to have ovarian cancer may indicate that the individual has ovarian cancer, thereby enabling detection of ovarian cancer at an early stage of the disease when treatment is most effective, perhaps before the ovarian cancer is detected by other means or before symptoms appear. Over-expression of one or more of the biomarkers during the course of ovarian cancer may be indicative of ovarian cancer progression, e.g., an ovarian tumor is growing and/or metastasizing (and thus indicate a poor prognosis), whereas a decrease in the degree to which one or more of the biomarkers is differentially expressed (i.e., in subsequent biomarker tests, the expression level in the individual is moving toward or approaching a “normal” expression level) may be indicative of ovarian cancer remission, e.g., an ovarian tumor is shrinking (and thus indicate a good or better prognosis). Similarly, an increase in the degree to which one or more of the biomarkers is differentially expressed (i.e., in subsequent biomarker tests, the expression level in the individual is moving further away from a “normal” expression level) during the course of ovarian cancer treatment may indicate that the ovarian cancer is progressing and therefore indicate that the treatment is ineffective, whereas a decrease in differential expression of one or more of the biomarkers during the course of ovarian cancer treatment may be indicative of ovarian cancer remission and therefore indicate that the treatment is working successfully. Additionally, an increase or decrease in the differential expression of one or more of the biomarkers after an individual has apparently been cured of ovarian cancer may be indicative of ovarian cancer recurrence. In a situation such as this, for example, the individual can be re-started on therapy (or the therapeutic regimen modified such as to increase dosage amount and/or frequency, if the individual has maintained therapy) at an earlier stage than if the recurrence of ovarian cancer was not detected until later. Furthermore, a differential expression level of one or more of the biomarkers in an individual may be predictive of the individual's response to a particular therapeutic agent. In monitoring for ovarian cancer recurrence or progression, changes in the biomarker expression levels may indicate the need for repeat imaging (e.g., repeat CT scanning), such as to determine ovarian cancer activity or to determine the need for changes in treatment.

Detection of any of the biomarkers described herein may be particularly useful following, or in conjunction with, ovarian cancer treatment, such as to evaluate the success of the treatment or to monitor ovarian cancer remission, recurrence, and/or progression (including metastasis) following treatment. Ovarian cancer treatment may include, for example, administration of a therapeutic agent to the individual, performance of surgery (e.g., surgical resection of at least a portion of an ovary or ovarian tumor), administration of radiation therapy, or any other type of ovarian cancer treatment used in the art, and any combination of these treatments. For example, any of the biomarkers may be detected at least once after treatment or may be detected multiple times after treatment (such as at periodic intervals), or may be detected both before and after treatment. Differential expression levels of any of the biomarkers in an individual over time may be indicative of ovarian cancer progression, remission, or recurrence, examples of which include any of the following: an increase or decrease in the expression level of the biomarkers after treatment compared with the expression level of the biomarker before treatment; an increase or decrease in the expression level of the biomarker at a later time point after treatment compared with the expression level of the biomarker at an earlier time point after treatment; and a differential expression level of the biomarker at a single time point after treatment compared with normal levels of the biomarker.

As a specific example, the biomarker levels for any of the biomarkers described herein can be determined in pre-surgery and post-surgery (e.g., 2-4 weeks after surgery) serum samples. An increase in the biomarker expression level(s) in the post-surgery sample compared with the pre-surgery sample can indicate progression of ovarian cancer (e.g., unsuccessful surgery), whereas a decrease in the biomarker expression level(s) in the post-surgery sample compared with the pre-surgery sample can indicate regression of ovarian cancer (e.g., the surgery successfully removed the tumor). Similar analyses of the biomarker levels can be carried out before and after other forms of treatment, such as before and after radiation therapy or administration of a therapeutic agent or cancer vaccine.

In addition to testing biomarker levels as a stand-alone diagnostic test, biomarker levels can also be done in conjunction with determination of SNPs or other genetic lesions or variability that are indicative of increased risk of susceptibility of disease. (See, e.g., Amos et al., Nature Genetics 40, 616-622 (2009)).

In addition to testing biomarker levels as a stand-alone diagnostic test, biomarker levels can also be done in conjunction with CT screening. For example, the biomarkers may facilitate the medical and economic justification for implementing CT screening, such as for screening large asymptomatic populations at risk for ovarian cancer. For example, a “pre-CT” test of biomarker levels could be used to stratify high-risk individuals for CT screening, such as for identifying those who are at highest risk for ovarian cancer based on their biomarker levels and who should be prioritized for CT screening. If a CT test is implemented, biomarker levels of one or more biomarkers can be measured and evaluated in conjunction with additional biomedical information (e.g., tumor parameters determined by CT testing) to enhance positive predictive value (PPV) over CT or biomarker testing alone. A “post-CT” panel for determining biomarker levels can be used to determine the likelihood that an ovarian nodule observed by CT (or other imaging modality) is malignant or benign.

Detection of any of the biomarkers described herein may be useful for post-CT testing. For example, biomarker testing may eliminate or reduce a significant number of false positive tests over CT alone. Further, biomarker testing may facilitate treatment of patients. By way of example, if a nodule is less than 5 mm in size, results of biomarker testing may advance patients from “watch and wait” to biopsy at an earlier time; if a nodule is 5-9 mm, biomarker testing may eliminate the use of a biopsy on false positive scans; and if a nodule is larger than 10 mm, biomarker testing may eliminate surgery for a sub-population of these patients with benign nodules. Eliminating the need for biopsy in some patients based on biomarker testing would be beneficial because there is significant morbidity associated with nodule biopsy and difficulty in obtaining nodule tissue depending on the location of nodule. Similarly, eliminating the need for surgery in some patients, such as those whose nodules are actually benign, would avoid unnecessary risks and costs associated with surgery.

In addition to testing biomarker levels in conjunction with CT screening (e.g., assessing biomarker levels in conjunction with size or other characteristics of a nodule observed on a CT scan), information regarding the biomarkers can also be evaluated in conjunction with other types of data, particularly data that indicates an individual's risk for ovarian cancer (e.g., patient clinical history, symptoms, family history of cancer, risk factors, and/or status of other biomarkers, etc.). These various data can be assessed by automated methods, such as a computer program/software, which can be embodied in a computer or other apparatus/device.

Any of the described biomarkers may also be used in imaging tests. For example, an imaging agent can be coupled to any of the described biomarkers, which can be used to aid in ovarian cancer diagnosis, to monitor disease progression/remission or metastasis, to monitor for disease recurrence, or to monitor response to therapy, among other uses.

B. Detection and Determination of Biomarkers and Biomarker Values

A biomarker level or value for the biomarkers described herein can be detected using any of a variety of known analytical methods. In one embodiment, a biomarker value is detected using a capture reagent. As used herein, a “capture agent” or “capture reagent” refers to a molecule that is capable of binding specifically to a biomarker. In various embodiments, the capture reagent can be exposed to the biomarker in solution or can be exposed to the biomarker while the capture reagent is immobilized on a solid support. In other embodiments, the capture reagent contains a feature that is reactive with a secondary feature on a solid support. In these embodiments, the capture reagent can be exposed to the biomarker in solution, and then the feature on the capture reagent can be used in conjunction with the secondary feature on the solid support to immobilize the biomarker on the solid support. The capture reagent is selected based on the type of analysis to be conducted. Capture reagents include but are not limited to aptamers, antibodies, adnectins, ankyrins, other antibody mimetics and other protein scaffolds, autoantibodies, chimeras, small molecules, an F(ab′)₂ fragment, a single chain antibody fragment, an Fv fragment, a single chain Fv fragment, a nucleic acid, a lectin, a ligand-binding receptor, affybodies, nanobodies, imprinted polymers, avimers, peptidomimetics, a hormone receptor, a cytokine receptor, and synthetic receptors, and modifications and fragments of these.

In some embodiments, a biomarker level or value is detected using a biomarker/capture reagent complex.

In other embodiments, the biomarker value is derived from the biomarker/capture reagent complex and is detected indirectly, such as, for example, as a result of a reaction that is subsequent to the biomarker/capture reagent interaction, but is dependent on the formation of the biomarker/capture reagent complex.

In some embodiments, the biomarker value is detected directly from the biomarker in a biological sample.

In one embodiment, the biomarkers are detected using a multiplexed format that allows for the simultaneous detection of two or more biomarkers in a biological sample. In one embodiment of the multiplexed format, capture reagents are immobilized, directly or indirectly, covalently or non-covalently, in discrete locations on a solid support. In another embodiment, a multiplexed format uses discrete solid supports where each solid support has a unique capture reagent associated with that solid support, such as, for example quantum dots. In another embodiment, an individual device is used for the detection of each one of multiple biomarkers to be detected in a biological sample. Individual devices can be configured to permit each biomarker in the biological sample to be processed simultaneously. For example, a microtiter plate can be used such that each well in the plate is used to uniquely analyze one of multiple biomarkers to be detected in a biological sample.

In one or more of the foregoing embodiments, a fluorescent tag can be used to label a component of the biomarker/capture complex to enable the detection of the biomarker value. In various embodiments, the fluorescent label can be conjugated to a capture reagent specific to any of the biomarkers described herein using known techniques, and the fluorescent label can then be used to detect the corresponding biomarker value. Suitable fluorescent labels include rare earth chelates, fluorescein and its derivatives, rhodamine and its derivatives, dansyl, allophycocyanin, PBXL-3, Qdot 605, Lissamine, phycoerythrin, Texas Red, and other such compounds.

In one embodiment, the fluorescent label is a fluorescent dye molecule. In some embodiments, the fluorescent dye molecule includes at least one substituted indolium ring system in which the substituent on the 3-carbon of the indolium ring contains a chemically reactive group or a conjugated substance. In some embodiments, the dye molecule includes an Alexa Fluor® molecule, such as, for example, Alexa Fluor® 488, Alexa Fluor® 532, Alexa Fluor® 647, Alexa Fluor® 680, or Alexa Fluor® 700. In other embodiments, the dye molecule includes a first type and a second type of dye molecule, such as, e.g., two different Alexa Fluor® molecules. In other embodiments, the dye molecule includes a first type and a second type of dye molecule, and the two dye molecules have different emission spectra.

Fluorescence can be measured with a variety of instrumentation compatible with a wide range of assay formats. For example, spectrofluorimeters have been designed to analyze microtiter plates, microscope slides, printed arrays, cuvettes, etc. See Principles of Fluorescence Spectroscopy, by J. R. Lakowicz, Springer Science+Business Media, Inc., 2004. See Bioluminescence & Chemiluminescence: Progress & Current Applications; Philip E. Stanley and Larry J. Kricka editors, World Scientific Publishing Company, January 2002.

A chemiluminescence tag can optionally be used to label a component of the biomarker/capture complex to enable the detection of a biomarker value. Suitable chemiluminescent materials include any of oxalyl chloride, Rodamin 6G, Ru(bipy)₃²⁺, TMAE (tetrakis(dimethylamino)ethylene), Pyrogallol (1,2,3-trihydroxibenzene), Lucigenin, peroxyoxalates, Aryl oxalates, Acridinium esters, dioxetanes, and others.

The detection method can include an enzyme/substrate combination that generates a detectable signal that corresponds to the biomarker value. Generally, the enzyme catalyzes a chemical alteration of the chromogenic substrate which can be measured using various techniques, including spectrophotometry, fluorescence, and chemiluminescence. Suitable enzymes include, for example, luciferases, luciferin, malate dehydrogenase, urease, horseradish peroxidase (HRPO), alkaline phosphatase, beta-galactosidase, glucoamylase, lysozyme, glucose oxidase, galactose oxidase, and glucose-6-phosphate dehydrogenase, uricase, xanthine oxidase, lactoperoxidase, microperoxidase, and the like.

The detection method can be a combination of fluorescence, chemiluminescence, radionuclide or enzyme/substrate combinations that generate a measurable signal. Multimodal signaling could have unique and advantageous characteristics in biomarker assay formats.

More specifically, the biomarker values for the biomarkers described herein can be detected using known analytical methods including, singleplex aptamer assays, multiplexed aptamer assays, singleplex or multiplexed immunoassays, mass spectrometric analysis, histological/cytological methods, etc. as detailed below.

C. Determination of Biomarker Values Using Immunoassays

Immunoassay methods are based on the reaction of an antibody to its corresponding target or analyte and can detect the analyte in a sample depending on the specific assay format. To improve specificity and sensitivity of an assay method based on immuno-reactivity, monoclonal antibodies are often used because of their specific epitope recognition. Polyclonal antibodies have also been successfully used in various immunoassays because of their increased affinity for the target as compared to monoclonal antibodies Immunoassays have been designed for use with a wide range of biological sample matrices Immunoassay formats have been designed to provide qualitative, semi-quantitative, and quantitative results.

Quantitative results are generated through the use of a standard curve created with known concentrations of the specific analyte to be detected. The response or signal from an unknown sample is plotted onto the standard curve, and a quantity or value corresponding to the target in the unknown sample is established.

Numerous immunoassay formats have been designed. ELISA or EIA can be quantitative for the detection of an analyte. This method relies on attachment of a label to either the analyte or the antibody and the label component includes, either directly or indirectly, an enzyme. ELISA tests may be formatted for direct, indirect, competitive, or sandwich detection of the analyte. Other methods rely on labels such as, for example, radioisotopes (I¹²⁵) or fluorescence. Additional techniques include, for example, agglutination, nephelometry, turbidimetry, Western blot, immunoprecipitation, immunocytochemistry, immunohistochemistry, flow cytometry, Luminex® assay, and others (see ImmunoAssay: A Practical Guide, edited by Brian Law, published by Taylor & Francis, Ltd., 2005 edition).

Exemplary assay formats include enzyme-linked immunosorbent assay (ELISA), radioimmunoassay, fluorescent, chemiluminescence, and fluorescence resonance energy transfer (FRET) or time resolved-FRET (TR-FRET) immunoassays. Examples of procedures for detecting biomarkers include biomarker immunoprecipitation followed by quantitative methods that allow size and peptide level discrimination, such as gel electrophoresis, capillary electrophoresis, planar electrochromatography, and the like.

Methods of detecting and/or quantifying a detectable label or signal generating material depend on the nature of the label. The products of reactions catalyzed by appropriate enzymes (where the detectable label is an enzyme; see above) can be, without limitation, fluorescent, luminescent, or radioactive or they may absorb visible or ultraviolet light. Examples of detectors suitable for detecting such detectable labels include, without limitation, x-ray film, radioactivity counters, scintillation counters, spectrophotometers, colorimeters, fluorometers, luminometers, and densitometers.

Any of the methods for detection can be performed in any format that allows for any suitable preparation, processing, and analysis of the reactions. This can be, for example, in multi-well assay plates (e.g., 96 wells or 384 wells) or using any suitable array or microarray. Stock solutions for various agents can be made manually or robotically, and all subsequent pipetting, diluting, mixing, distribution, washing, incubating, sample readout, data collection and analysis can be done robotically using commercially available analysis software, robotics, and detection instrumentation capable of detecting a detectable label.

E. Determination of Biomarker Values Using Mass Spectrometry Methods

A variety of configurations of mass spectrometers can be used to detect biomarker values. Several types of mass spectrometers are available or can be produced with various configurations. In general, a mass spectrometer has the following major components: a sample inlet, an ion source, a mass analyzer, a detector, a vacuum system, and instrument-control system, and a data system. Difference in the sample inlet, ion source, and mass analyzer generally define the type of instrument and its capabilities. For example, an inlet can be a capillary-column liquid chromatography source or can be a direct probe or stage such as used in matrix-assisted laser desorption. Common ion sources are, for example, electrospray, including nanospray and microspray or matrix-assisted laser desorption. Common mass analyzers include a quadrupole mass filter, ion trap mass analyzer and time-of-flight mass analyzer. Additional mass spectrometry methods are well known in the art (see Burlingame et al. Anal. Chem. 70:647R-716R (1998); Kinter and Sherman, New York (2000)).

Protein biomarkers and biomarker values can be detected and measured by any of the following: electrospray ionization mass spectrometry (ESI-MS), ESI-MS/MS, ESI-MS/(MS)n, matrix-assisted laser desorption ionization time-of-flight mass spectrometry (MALDI-TOF-MS), surface-enhanced laser desorption/ionization time-of-flight mass spectrometry (SELDI-TOF-MS), desorption/ionization on silicon (DIOS), secondary ion mass spectrometry (SIMS), quadrupole time-of-flight (Q-TOF), tandem time-of-flight (TOF/TOF) technology, called ultraflex III TOF/TOF, atmospheric pressure chemical ionization mass spectrometry (APCI-MS), APCI-MS/MS, APCI-(MS).sup.N, atmospheric pressure photoionization mass spectrometry (APPI-MS), APPI-MS/MS, and APPI-(MS)^{N}, quadrupole mass spectrometry, Fourier transform mass spectrometry (FTMS), quantitative mass spectrometry, and ion trap mass spectrometry.

Sample preparation strategies are used to label and enrich samples before mass spectroscopic characterization of protein biomarkers and determination biomarker values. Labeling methods include but are not limited to isobaric tag for relative and absolute quantitation (iTRAQ) and stable isotope labeling with amino acids in cell culture (SILAC). Capture reagents used to selectively enrich samples for candidate biomarker proteins prior to mass spectroscopic analysis include but are not limited to aptamers, antibodies, nucleic acid probes, chimeras, small molecules, an F(ab′)₂ fragment, a single chain antibody fragment, an Fv fragment, a single chain Fv fragment, a nucleic acid, a lectin, a ligand-binding receptor, affybodies, nanobodies, ankyrins, domain antibodies, alternative antibody scaffolds (e.g. diabodies etc) imprinted polymers, avimers, peptidomimetics, peptoids, peptide nucleic acids, threose nucleic acid, a hormone receptor, a cytokine receptor, and synthetic receptors, and modifications and fragments of these.

The foregoing assays enable the detection of biomarker values that are useful in methods for diagnosing ovarian cancer, where the methods comprise detecting, in a biological sample from an individual, at least one biomarker value that corresponds to a biomarker selected from the group consisting of the biomarkers provided in Table 1. Various embodiments provide combinations of multiple biomarkers as described in FIGS. 2A-6AC. In another aspect, methods are provided for determining remission of ovarian cancer by detecting, in a biological sample from an individual, at least one of the biomarkers provided in Table 1.

### EXAMPLES

### Methods and Materials

### Human Subjects and Serum Samples

This study was approved by the institutional review board of the Georgia Health Sciences University and informed consent was obtained from every subject or a legally authorized representative. The subjects used in this study included 106 ovarian cancer patients and 232 healthy women as control. Disease progression was defined by either CA125 levels≧2×nadir value on two occasions, increase in lesions or death (18). Patient's conditions were staged according to the criteria of the International Federation of Gynecology and Obstetrics (FIGO). The age distribution and tumor characteristics of the patient population are presented in Supplementary Table 1. A total of 150 serum samples from 106 patients were obtained at 3 different stages of disease progression: post-diagnosis (PD, n=46), remission (RM, n=51) and recurrent (RC, n=53).

### Luminex® Assays

The Luminex® kits were obtained from Millipore (Billerica, Mass., USA) and assays were performed as per manufacturer's instructions to determine the serum levels of 46 molecules. Properly diluted serum samples were incubated with the antibody-coupled microspheres and then with biotinylated detection antibody before the addition of streptavidin-phycoerythrin. The captured bead-complexes were measured with FLEXMAP 3D system (Luminex Corporation, Austin, Tex., USA).

### Statistical Analyses

All statistical analyses were performed using the R language and environment for statistical computing (R version 2.12.1; R Foundation for Statistical Computing). We used both single protein and multi-marker models for the classification of cases and controls. Linear discriminate analysis was performed using combinations of 3 to 5 protein models. The performance of each model was evaluated using the leave-one-out cross validation method. The statistical comparison of the area-under-the-curve (AUC) of the receiver-operating-characteristic (ROC) curves for different models was performed using Wilcoxon statistic. We used Cox proportional hazards models to evaluate the impact of serum protein levels on survival. Overall survival was calculated as time from diagnosis date to the death of patient. Patients who are alive with no evidence of disease were censored at the date of last follow-up visit. Univariate analyses were performed by using the Kaplan-Meier plots, and statistical significance between survival curves was assessed using the log rank test. To assess the combined effect of different proteins on survival, multivariate analysis was performed using proteins having significant effect in the univariate analysis.

### Example 1

### Twenty-Eight Proteins are Altered in OC

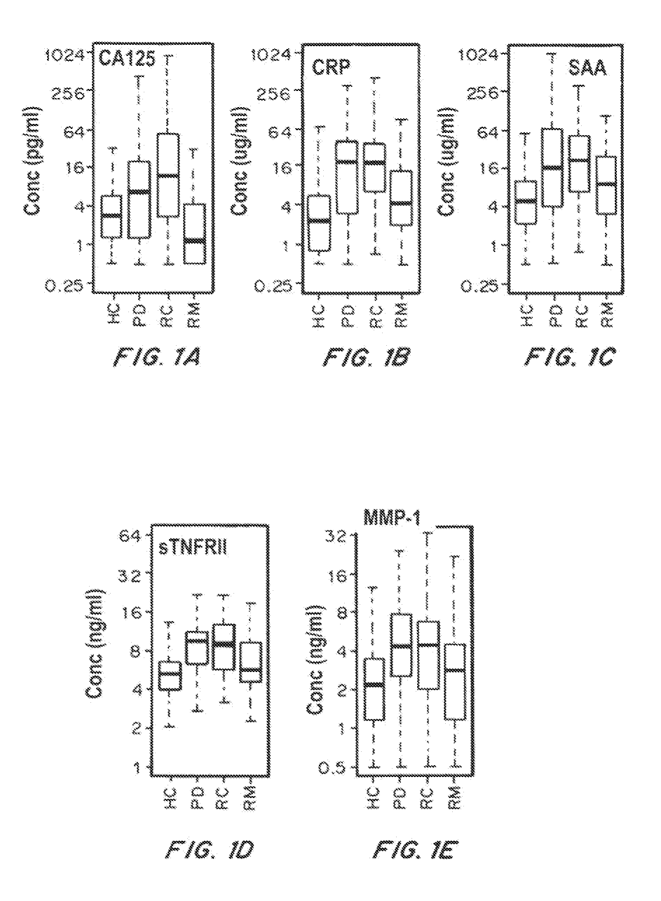

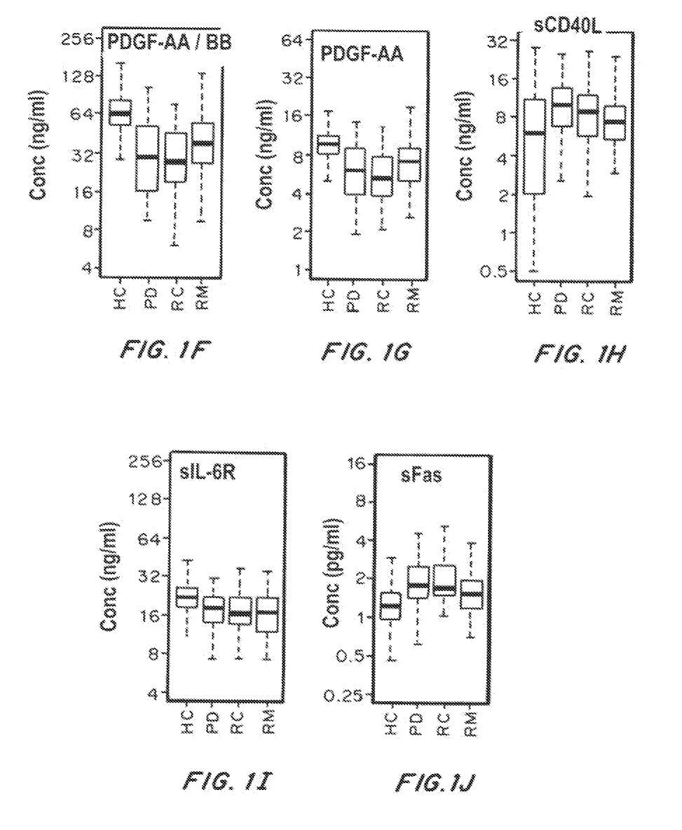

Serum levels of 40 serum proteins were analyzed as described above. While 40 proteins could be accurately measured in the majority of the samples, >30% of the samples had levels below the limit of detection for 5 proteins (AFP, CA19-9, SCCA, CYFRA21-1, and sFasL). These 5 proteins were excluded from subsequent analyses. Serum levels of the 40 proteins in the PD, RC, and RM groups were compared with healthy controls (HC) using a student's t-test. Significant differences were found for 28 proteins in at least one of the three groups as compared to the HC group (Table 1 and Supp Table 2). Box plots for ten representative proteins are shown in FIGS. 1A-1J.

The most highly increased proteins are CRP and SAA, suggesting active inflammation in the patients with active disease (PD and RC) but to a lesser degree at the remission stage. Inflammation in OC is also indicated by the increased levels of soluble receptors such as sTNFR-II and sCD40L. Many of these molecules (CRP, SAA, sTNFR-II, IGFBP-2, Leptin, CD40L and sFAS) have previously been reported in OC (9, 19-22, 29-34).

The most down-regulated proteins are PDGF-AA/BB and PDGF-AA, two related molecules which play a critical role in cell proliferation and angiogenesis. Genomic studies suggested that activation of the PDGF pathway plays a critical role in OC (35). While the pro-angiogenic and pro-growth function of PDGF would predict higher levels of serum PDGF (36), these two proteins are surprisingly lower in OC patients compared to HC. Consistent with our results, PDGF-AA was also reported to be significantly lower in sera of pancreatic cancer patients (37).

TABLE 1
Significant changes in serum protein levels in patients as compared
to healthy controls (PD: Post Diagnosis, HC: Healthy Controls,
RC: Recurrence, RM: Remission)

| Protein | PD/HC | p-val | RC/HC | p-val | RM/HC | p-val |
|---|---|---|---|---|---|---|
| PDGF-AA/BB | 0.45 | 8e−10 | 0.42 | 7e−12 | 0.54 | 3e−07 |
| sCD40L | 2.21 | 6e−09 | 1.76 | 4e−05 | 1.61 | 5e−04 |
| PDGF-AA | 0.64 | 9e−08 | 0.55 | 4e−11 | 0.68 | 7e−06 |
| CRP | 5.01 | 3e−06 | 6.35 | 7e−10 | 1.99 | 0.004 |
| SAA | 3.93 | 3e−05 | 4.83 | 2e−07 | 1.68 | 0.016 |
| MMP-1 | 2.00 | 3e−05 | 1.78 | 0.001 | 1.13 | 0.415 |
| IGFBP-2 | 2.69 | 2e−04 | 1.36 | 0.328 | 1.00 | 0.991 |
| CA125 | 2.35 | 0.004 | 4.71 | 3e−06 | 0.73 | 0.133 |
| Leptin | 1.61 | 0.075 | 2.46 | 5e−05 | 2.71 | 3e−07 |
| sTNFR-II | 1.65 | 6e−07 | 1.70 | 4e−08 | 1.24 | 0.010 |
| sFas | 1.47 | 7e−05 | 1.51 | 1e−08 | 1.26 | 0.002 |
| sIL-2Ra | 1.47 | 1e−04 | 1.22 | 0.043 | 1.05 | 0.614 |
| CD14 | 1.24 | 2e−04 | 1.25 | 7e−04 | 1.08 | 0.148 |
| sIL-6R | 0.80 | 2e−04 | 0.75 | 1e−06 | 0.74 | 9e−06 |
| IGFBP-6 | 1.42 | 4e−04 | 1.37 | 0.001 | 1.08 | 0.526 |
| tPAI-1 | 0.81 | 0.005 | 0.72 | 7e−05 | 0.72 | 5e−05 |
| HGF | 1.29 | 0.006 | 1.28 | 0.002 | 1.16 | 0.179 |
| sVCAM-1 | 0.86 | 0.012 | 0.79 | 1e−05 | 0.83 | 0.002 |
| sE-SELECTIN | 0.79 | 0.013 | 0.77 | 0.004 | 0.81 | 0.050 |
| MDC | 0.83 | 0.023 | 0.73 | 8e−04 | 0.85 | 0.038 |
| IGFBP-3 | 0.83 | 0.050 | 0.91 | 0.238 | 0.81 | 0.026 |
| MMP-2 | 0.87 | 0.108 | 0.80 | 0.002 | 0.77 | 0.004 |
| sIL-4R | 0.87 | 0.117 | 0.68 | 1e−05 | 0.81 | 0.030 |
| CEA | 1.23 | 0.157 | 1.36 | 0.055 | 0.94 | 0.590 |
| CA15-3 | 1.21 | 0.184 | 1.47 | 0.006 | 1.03 | 0.828 |
| sICAM-1 | 1.07 | 0.315 | 0.96 | 0.547 | 0.82 | 0.005 |
| sgp130 | 0.94 | 0.345 | 0.85 | 0.006 | 0.82 | 0.003 |
| MMP-9 | 1.38 | 0.047 | 1.25 | 0.108 | 1.31 | 0.064 |

SUPPLEMENTARY TABLE 1
Characteristics of the patient population

| | Control | PD | RC | RM |
|---|---|---|---|---|
| | (n = 232) | (n = 47) | (n = 53) | (n = 50) |
| Age(year) | | | | |
| Mean ± SD | 48.77 ± | 61.10 ± | 63.22 ± | 59.83 ± |
| | 10.12 | 12.60 | 12.31 | 14.35 |
| Median | 47.55 | 60.38 | 64.18 | 60.23 |
| Range | (27.36- | (36.28- | (39.10- | (27.48- |
| | 80.33) | 87.24) | 89.26) | 95.79) |
| FIGO staging | | | | |
| Stage I | | 2 | 7 | 19 |
| Stage II | | 4 | 4 | 7 |
| Stage III | | 33 | 39 | 22 |
| Stage IV | | 8 | 3 | 2 |
| Histological | | | | |
| type | | | | |
| Serous | | 31 | 42 | 35 |
| Mucinous | | 0 | 1 | 2 |
| Endometrioid | | 5 | 3 | 6 |
| Clear cell | | 1 | 2 | 2 |
| mixed | | 7 | 1 | 1 |
| others | | 3 | 4 | 4 |
| Tumor grade | | | | |
| Grade 1 | | 0 | 7 | 12 |
| Grade 2 | | 10 | 12 | 8 |
| Grade 3 | | 37 | 34 | 30 |
| Surgery type | | | | |
| optimal | | 26 | 35 | 40 |
| suboptimal | | 21 | 19 | 10 |

A total of 150 serum samples from 106 patients were obtained at 3 different stages of disease progression: post-diagnosis (PD, n=46), remission (RM, n=51) and recurrent (RC, n=53).

SUPPLEMENTARY TABLE 2
Changes in serum protein levels in patients as
compared to healthy controls (PD: Post Diagnosis, HC: Healthy Controls,
RC: Recurrence, RM: Remission)

| Protein | PD/HC | p-val | RC/HC | p-val | RM/HC | p-val |
|---|---|---|---|---|---|---|
| PDGF-AA/BB | 0.45 | 8e−10 | 0.42 | 7e−12 | 0.54 | 3e−07 |
| SCD40L | 2.21 | 6e−09 | 1.76 | 4e−05 | 1.61 | 5e−04 |
| PDGF-AA | 0.64 | 9e−08 | 0.55 | 4e−11 | 0.68 | 7e−06 |
| CRP | 5.01 | 3e−06 | 6.35 | 7e−10 | 1.99 | 0.004 |
| SAA | 3.93 | 3e−05 | 4.83 | 2e−07 | 1.68 | 0.016 |
| MMP-1 | 2.00 | 3e−05 | 1.78 | 0.001 | 1.13 | 0.415 |
| IGFBP-2 | 2.69 | 2e−04 | 1.36 | 0.328 | 1.00 | 0.991 |
| CA125 | 2.35 | 0.004 | 4.71 | 3e−06 | 0.73 | 0.133 |
| Leptin | 1.61 | 0.075 | 2.46 | 5e−05 | 2.71 | 3e−07 |
| sTNFR-I | 2.52 | 2e−17 | 2.12 | 2e−08 | 1.71 | 1e−05 |
| sTNFR-II | 1.65 | 6e−07 | 1.70 | 4e−08 | 1.24 | 0.010 |
| sFas | 1.47 | 7e−05 | 1.51 | 1e−08 | 1.26 | 0.002 |
| sIL-2Ra | 1.47 | 1e−04 | 1.22 | 0.043 | 1.05 | 0.614 |
| CD14 | 1.24 | 2e−04 | 1.25 | 7e−04 | 1.08 | 0.148 |
| SIL-6R | 0.80 | 2e−04 | 0.75 | 1e−06 | 0.74 | 9e−06 |
| IGFBP-6 | 1.42 | 4e−04 | 1.37 | 0.001 | 1.08 | 0.526 |
| tPAI-1 | 0.81 | 0.005 | 0.72 | 7e−05 | 0.72 | 5e−05 |
| HGF | 1.29 | 0.006 | 1.28 | 0.002 | 1.16 | 0.179 |
| sVCAM-1 | 0.86 | 0.012 | 0.79 | 1e−05 | 0.83 | 0.002 |
| sE-SELECTIN | 0.79 | 0.013 | 0.77 | 0.004 | 0.81 | 0.050 |
| MDC | 0.83 | 0.023 | 0.73 | 8e−04 | 0.85 | 0.038 |
| IGFBP-3 | 0.83 | 0.050 | 0.91 | 0.238 | 0.81 | 0.026 |
| MMP-2 | 0.87 | 0.108 | 0.80 | 0.002 | 0.77 | 0.004 |
| sIL-4R | 0.87 | 0.117 | 0.68 | 1e−05 | 0.81 | 0.030 |
| CEA | 1.23 | 0.157 | 1.35 | 0.055 | 0.94 | 0.590 |
| CA15-3 | 1.21 | 0.184 | 1.47 | 0.006 | 1.03 | 0.828 |
| sICAM-1 | 1.07 | 0.315 | 0.96 | 0.547 | 0.82 | 0.005 |
| sgp130 | 0.94 | 0.345 | 0.85 | 0.006 | 0.82 | 0.003 |
| MMP-9 | 1.38 | 0.047 | 1.25 | 0.108 | 1.31 | 0.064 |
| OPN | 1.23 | 0.086 | 1.14 | 0.450 | 0.90 | 0.459 |
| sEGFR | 1.15 | 0.097 | 0.97 | 0.769 | 0.93 | 0.459 |
| TPO | 1.27 | 0.195 | 1.18 | 0.352 | 1.13 | 0.570 |
| IGFBP-7 | 1.11 | 0.263 | 1.13 | 0.153 | 0.96 | 0.682 |
| OPG | 1.12 | 0.300 | 1.08 | 0.454 | 0.92 | 0.582 |
| PTH | 1.17 | 0.370 | 1.03 | 0.852 | 0.95 | 0.817 |
| MIG | 0.89 | 0.391 | 0.96 | 0.804 | 0.76 | 0.083 |
| GRO | 1.06 | 0.579 | 1.06 | 0.527 | 0.92 | 0.439 |
| IGFBP-1 | 1.12 | 0.609 | 1.06 | 0.762 | 1.10 | 0.659 |
| RANTES | 1.04 | 0.758 | 0.98 | 0.832 | 0.82 | 0.109 |
| sIL-1RII | 1.02 | 0.774 | 0.90 | 0.264 | 0.88 | 0.276 |
| MCP-1 | 1.00 | 0.995 | 0.92 | 0.323 | 0.92 | 0.332 |

### Example 2

### Protein Panels Accurately Distinguish Active Cancer from Controls

The utility of serum proteins as OC biomarkers was initially evaluated using AUC values. The top 10 molecules that can distinguish cancer (PD+RC) from HC are shown in FIGS. 2A-2J. The two best performing molecules are PDGF-AA/BB (AUC=0.85) and PDGF-AA (AUC=0.82). CRP and SAA also have excellent AUC (0.76 and 0.72, respectively). Interestingly, CA125 only has an AUC value of 0.65 in this dataset.

It is well known that combinations of molecules may significantly improve the performance of biomarkers. Groups of 3 proteins were analyzed to minimize the overfitting concern. AUC values were calculated for all possible three-marker combinations with the 40 serum proteins reliably measured in this study and found 131 models with AUC greater than 0.90 (Supp. Table 4). The top 10 most frequent molecules appearing in these 131 models include sCD40L, PDGF-AA/BB, PDGF-AA, CRP, MMP-I, sTNFR-II, sIL-6R, SAA, MMP-9 and CA125 (Supp. Table 5). The AUC of the individual proteins was in the range of 0.645 to 0.849 (FIGS. 2A-2J). The top ten three-marker models are illustrated in FIGS. 2K-2T. The best model (PDGFAA/BB+CRP+sCD40L) has an AUC value of 0.94) and ten models have AUC values greater than 0.92, significantly better than the two best individual proteins (AUC=0.85 for PDGF-AA/BB and AUC=0.82 for PDGF-AA).

SUPPLEMENTARY TABLE 4
131 Models (03 molecules in each model) with AUC >0.9
Multivariate analysis was performed for classification of
healthy controls and patients (HC vs PD + RC)

| No. | Mol1 | Mol2 | Mol3 | AUC |
|---|---|---|---|---|
| 1 | CRP | PDGF.AABB | sCD40L | 0.940 |
| 2 | CRP | PDGF.AA | sCD40L | 0.936 |
| 3 | PDGF.AABB | SAA | sCD40L | 0.934 |
| 4 | PDGF.AA | sCD40L | sTNFRII | 0.933 |
| 5 | MMP.1 | PDGF.AABB | sCD40L | 0.932 |
| 6 | PDGF.AA | SAA | sCD40L | 0.93 |
| 7 | PDGF.AA | sCD40L | sIL.6R | 0.93 |
| 8 | PDGF.AABB | sCD40L | sTNFRII | 0.929 |
| 9 | MMP.1 | PDGF.AA | sCD40L | 0.928 |
| 10 | PDGF.AA | PDGF.AABB | sCD40L | 0.927 |
| 11 | MMP.2 | PDGF.AA | sCD40L | 0.926 |
| 12 | IGFBP3 | PDGF.AA | sCD40L | 0.924 |
| 13 | PDGF.AABB | sCD40L | sIL.6R | 0.924 |
| 14 | GRO | PDGF.AA | sCD40L | 0.923 |
| 15 | CRP | PDGF.AABB | sIL.6R | 0.92 |
| 16 | PDGF.AABB | sCD40L | sIL.1RII | 0.92 |
| 17 | CEA | PDGF.AABB | sCD40L | 0.919 |
| 18 | PDGF.AA | sCD40L | sVCAM.1 | 0.919 |
| 19 | PDGF.AABB | RANTES | sCD40L | 0.919 |
| 20 | PDGF.AABB | sCD40L | sFas | 0.919 |
| 21 | PDGF.AABB | sIL.6R | sTNFRII | 0.919 |
| 22 | CD14 | CRP | PDGF.AABB | 0.918 |
| 23 | CA125 | PDGF.AABB | sCD40L | 0.918 |
| 24 | CEA | PDGF.AA | sCD40L | 0.918 |
| 25 | CRP | PDGF.AABB | sFas | 0.918 |
| 26 | HGF | PDGF.AABB | sCD40L | 0.918 |
| 27 | MDC | PDGF.AA | sCD40L | 0.918 |
| 28 | MMP.9 | PDGF.AA | sCD40L | 0.918 |
| 29 | PDGF.AA | sCD40L | sE.SELECTIN | 0.918 |
| 30 | PDGF.AA | sIL.6R | sTNFRII | 0.917 |
| 31 | CA125 | MMP.1 | PDGF.AABB | 0.916 |
| 32 | CA15.3 | PDGF.AA | sCD40L | 0.916 |
| 33 | CEA | CRP | PDGF.AABB | 0.916 |
| 34 | CRP | PDGF.AABB | sE.SELECTIN | 0.916 |
| 35 | HGF | PDGF.AA | sCD40L | 0.916 |
| 36 | IGFBP.1 | PDGF.AABB | sCD40L | 0.916 |
| 37 | PDGF.AA | sCD40L | sFas | 0.916 |
| 38 | CD14 | PDGF.AABB | sCD40L | 0.915 |
| 39 | CRP | MMP.1 | PDGF.AABB | 0.915 |
| 40 | CRP | PDGF.AABB | sTNFRII | 0.915 |
| 41 | MMP.2 | PDGF.AABB | sCD40L | 0.915 |
| 42 | MMP.9 | PDGF.AABB | sCD40L | 0.915 |
| 43 | PDGF.AA | RANTES | sCD40L | 0.915 |
| 44 | PDGF.AA | sCD40L | sIL.4R | 0.915 |
| 45 | PDGF.AABB | sCD40L | sICAM.1 | 0.915 |
| 46 | CA125 | CRP | PDGF.AABB | 0.914 |
| 47 | GRO | PDGF.AABB | sCD40L | 0.914 |
| 48 | Leptin | PDGF.AABB | sCD40L | 0.914 |
| 49 | PDGF.AA | sCD40L | sIL.1RII | 0.914 |
| 50 | CA15.3 | PDGF.AABB | sCD40L | 0.913 |
| 51 | CRP | PDGF.AA | PDGF.AABB | 0.913 |
| 52 | PDGF.AA | sCD40L | sgp130 | 0.913 |
| 53 | CD14 | MMP.1 | PDGF.AABB | 0.912 |
| 54 | IGFBP.1 | PDGF.AA | sCD40L | 0.912 |
| 55 | IGFBP.6 | PDGF.AA | sCD40L | 0.912 |
| 56 | IGFBP.6 | PDGF.AABB | sCD40L | 0.912 |
| 57 | OPN | PDGF.AABB | sCD40L | 0.912 |
| 58 | PDGF.AABB | sCD40L | sIL.4R | 0.912 |
| 59 | PDGF.AABB | sCD40L | sVCAM.1 | 0.912 |
| 60 | CD14 | PDGF.AA | sCD40L | 0.911 |
| 61 | CRP | PDGF.AA | sE.SELECTIN | 0.911 |
| 62 | IGFBP.7 | PDGF.AABB | sCD40L | 0.911 |
| 63 | MDC | PDGF.AABB | sCD40L | 0.911 |
| 64 | MIG | PDGF.AABB | sCD40L | 0.911 |
| 65 | OPG | PDGF.AA | sCD40L | 0.911 |
| 66 | PDGF.AA | PTH | sCD40L | 0.911 |
| 67 | PDGF.AABB | PTH | sCD40L | 0.911 |
| 68 | PDGF.AABB | sCD40L | sE.SELECTIN | 0.911 |
| 69 | CA15.3 | CRP | PDGF.AABB | 0.91 |
| 70 | CRP | HGF | PDGF.AABB | 0.91 |
| 71 | CRP | PDGF.AABB | sEGFR | 0.91 |
| 72 | IGFBP3 | PDGF.AABB | sCD40L | 0.91 |
| 73 | IGFBP.7 | PDGF.AA | sCD40L | 0.91 |
| 74 | MIG | PDGF.AA | sCD40L | 0.91 |
| 75 | MMP.1 | PDGF.AABB | SAA | 0.91 |
| 76 | MMP.1 | PDGF.AABB | sTNFRII | 0.91 |
| 77 | OPG | PDGF.AABB | sCD40L | 0.91 |
| 78 | PDGF.AA | sCD40L | sEGFR | 0.91 |
| 79 | PDGF.AA | sCD40L | TPO | 0.91 |
| 80 | MCP.1 | PDGF.AA | sCD40L | 0.909 |
| 81 | OPN | PDGF.AA | sCD40L | 0.909 |
| 82 | PDGF.AA | sCD40L | sICAM.1 | 0.909 |
| 83 | PDGF.AA | sTNFRII | sVCAM.1 | 0.909 |
| 84 | PDGF.AABB | sCD40L | sgp130 | 0.909 |
| 85 | PDGF.AABB | sCD40L | TPO | 0.909 |
| 86 | CA125 | PDGF.AABB | sTNFRII | 0.908 |
| 87 | CRP | PDGF.AABB | SAA | 0.908 |
| 88 | CRP | PDGF.AABB | sIL.1RII | 0.908 |
| 89 | IGFBP.2 | PDGF.AA | sCD40L | 0.908 |
| 90 | Leptin | PDGF.AA | sCD40L | 0.908 |
| 91 | MCP.1 | PDGF.AABB | sCD40L | 0.908 |
| 92 | PDGF.AA | sCD40L | tPAI.1 | 0.908 |
| 93 | PDGF.AABB | sCD40L | sEGFR | 0.908 |
| 94 | PDGF.AABB | sCD40L | sIL.2Ra | 0.908 |
| 95 | CRP | GRO | PDGF.AABB | 0.907 |
| 96 | CRP | MIG | PDGF.AABB | 0.907 |
| 97 | CRP | PDGF.AABB | sIL.2Ra | 0.907 |
| 98 | IGFBP.2 | PDGF.AABB | sCD40L | 0.907 |
| 99 | MMP.2 | PDGF.AA | sTNFRII | 0.907 |
| 100 | PDGF.AABB | sCD40L | tPAI.1 | 0.907 |
| 101 | CA125 | PDGF.AA | sCD40L | 0.906 |
| 102 | CRP | Leptin | PDGF.AABB | 0.906 |
| 103 | CRP | PDGF.AABB | RANTES | 0.906 |
| 104 | CRP | PDGF.AABB | tPAI.1 | 0.906 |
| 105 | IGFBP3 | MMP.1 | PDGF.AA | 0.906 |
| 106 | MMP.1 | PDGF.AABB | sFas | 0.906 |
| 107 | CRP | IGFBP.2 | PDGF.AABB | 0.905 |
| 108 | PDGF.AA | sCD40L | sIL.2Ra | 0.905 |
| 109 | PDGF.AABB | SAA | sIL.6R | 0.905 |
| 110 | CA125 | PDGF.AABB | SAA | 0.904 |
| 111 | CRP | MMP.9 | PDGF.AABB | 0.904 |
| 112 | CRP | sCD40L | tPAI.1 | 0.904 |
| 113 | PDGF.AABB | SAA | sTNFRII | 0.904 |
| 114 | CD14 | MMP.9 | PDGF.AABB | 0.903 |
| 115 | CRP | PDGF.AA | sTNFRII | 0.903 |
| 116 | MMP.1 | PDGF.AA | PDGF.AABB | 0.903 |
| 117 | sCD40L | sIL.6R | sTNFRII | 0.903 |
| 118 | CRP | MMP.9 | PDGF.AA | 0.902 |
| 119 | GRO | PDGF.AA | sTNFRII | 0.902 |
| 120 | HGF | MMP.1 | PDGF.AABB | 0.902 |
| 121 | MMP.1 | PDGF.AA | sTNFRII | 0.902 |
| 122 | CRP | MMP.2 | PDGF.AA | 0.901 |
| 123 | CRP | PDGF.AA | sVCAM.1 | 0.901 |
| 124 | Leptin | MMP.1 | PDGF.AABB | 0.901 |
| 125 | MMP.1 | MMP.2 | PDGF.AA | 0.901 |
| 126 | MMP.1 | PDGF.AABB | sIL.6R | 0.901 |
| 127 | MMP.9 | PDGF.AABB | RANTES | 0.901 |
| 128 | CRP | IGFBP3 | PDGF.AA | 0.9 |
| 129 | CRP | MMP.2 | PDGF.AABB | 0.9 |
| 130 | CRP | PDGF.AABB | sIL.4R | 0.9 |
| 131 | GRO | PDGF.AABB | sTNFRII | 0.9 |

SUPPLEMENTARY TABLE 5
Ten molecules selected as best classifiers
Multivariate analysis was performed for classification of healthy controls
and patients (HC vs PD + RC) using multivariate models
(03 molecules in each model)

| S. | | AUC | AUC | AUC | AUC | AUC | AUC | |
|---|---|---|---|---|---|---|---|---|
| No. | Molecule | >0.85 | >0.86 | >0.87 | >0.88 | >0.89 | >0.90 | Total |
| 1 | SCD40L | 85 | 83 | 79 | 78 | 77 | 77 | 479 |
| 2 | PDGF- | 407 | 340 | 242 | 171 | 127 | 76 | 1363 |
| | AA/BB | | | | | | | |
| 3 | PDGF-AA | 346 | 268 | 195 | 140 | 90 | 52 | 1091 |
| 4 | CRP | 92 | 82 | 77 | 77 | 65 | 30 | 423 |
| 5 | MMP-1 | 77 | 76 | 76 | 66 | 39 | 15 | 349 |
| 6 | sTNFR-II | 116 | 114 | 87 | 58 | 29 | 14 | 418 |
| 7 | sIL-6R | 105 | 74 | 47 | 28 | 12 | 8 | 274 |
| 8 | SAA | 80 | 78 | 60 | 31 | 23 | 7 | 279 |
| 9 | MMP-9 | 76 | 76 | 46 | 23 | 13 | 6 | 240 |
| 10 | CA125 | 51 | 33 | 28 | 19 | 7 | 6 | 144 |
| 11 | CD14 | 38 | 25 | 18 | 12 | 9 | 5 | 107 |
| 12 | MMP-2 | 36 | 23 | 17 | 9 | 7 | 5 | 97 |
| 13 | HGF | 66 | 64 | 34 | 15 | 6 | 4 | 189 |
| 14 | RANTES | 64 | 51 | 24 | 15 | 8 | 4 | 166 |
| 15 | sFas | 65 | 40 | 24 | 18 | 9 | 4 | 160 |
| 16 | GRO | 59 | 39 | 22 | 13 | 7 | 4 | 144 |
| 17 | tPAI-1 | 46 | 29 | 16 | 11 | 5 | 4 | 111 |
| 18 | Leptin | 37 | 27 | 18 | 13 | 6 | 4 | 105 |
| 19 | sE.SELECTIN | 30 | 25 | 16 | 10 | 9 | 4 | 94 |
| 20 | sVCAM-1 | 27 | 20 | 14 | 9 | 6 | 4 | 80 |

### Example 3

### Serum Profile at Remission is Distinct from Both Active Cancer and Controls

Seventeen proteins were significantly different between RM and HC (Table 1) while 15 proteins showed significant differences between RM and active cancer (PD or RC) (Supp Table 3). The mean level of CA125 in RM samples is significantly reduced and similar to the value in HC, while the RC group has the highest mean CA125 (Table 1 and Supp Table 3). These results further validate CA125 as a good marker for monitoring ovarian cancer. IGFBP2 in RM samples was also significantly reduced and returned to normal levels. Furthermore, the levels for CRP and SAA were also significantly reduced in the RM samples compared to both the PD and RC samples (Supp Table 3).

SUPPLEMENTARY TABLE 3
Changes in serum protein levels in patients as compared to Remission
cases (PD: Post Diagnosis, RC: Recurrence, RM: Remission)

| Protein | PD/RM | p-val | RC/RM | p-val |
|---|---|---|---|---|
| CA125 | 3.24 | 0.001 | 6.48 | 6e−07 |
| CRP | 2.51 | 0.013 | 3.18 | 5e−04 |
| SAA | 2.34 | 0.016 | 2.87 | 0.001 |
| sTNFR-I | 1.48 | 0.002 | 1.24 | 0.133 |
| sTNFR-II | 1.33 | 0.014 | 1.37 | 0.006 |
| MMP-1 | 1.77 | 0.007 | 1.57 | 0.043 |
| IGFBP-6 | 1.31 | 0.038 | 1.27 | 0.064 |
| sICAM-1 | 1.31 | 0.003 | 1.17 | 0.075 |
| OPN | 1.38 | 0.039 | 1.27 | 0.227 |
| sIL-2Ra | 1.39 | 0.014 | 1.15 | 0.288 |
| IGFBP-2 | 2.68 | 0.006 | 1.36 | 0.447 |
| sCD40L | 1.37 | 0.020 | 1.09 | 0.529 |
| PDGF-AA | 0.93 | 0.478 | 0.80 | 0.031 |
| CA15-3 | 1.17 | 0.349 | 1.42 | 0.035 |
| sFas | 1.17 | 0.146 | 1.20 | 0.035 |
| CEA | 1.31 | 0.124 | 1.44 | 0.049 |
| CD14 | 1.15 | 0.052 | 1.16 | 0.064 |
| PDGF-AA/BB | 0.84 | 0.250 | 0.78 | 0.090 |
| sIL-4R | 1.07 | 0.588 | 0.83 | 0.117 |
| MDC | 0.98 | 0.817 | 0.86 | 0.150 |
| MIG | 1.17 | 0.364 | 1.27 | 0.176 |
| IGFBP-7 | 1.16 | 0.247 | 1.18 | 0.180 |
| RANTES | 1.27 | 0.133 | 1.19 | 0.249 |
| IGFBP-3 | 1.03 | 0.789 | 1.13 | 0.294 |
| GRO | 1.16 | 0.335 | 1.16 | 0.305 |
| OPG | 1.22 | 0.244 | 1.18 | 0.329 |
| sVCAM-1 | 1.04 | 0.581 | 0.95 | 0.428 |
| HGF | 1.11 | 0.427 | 1.10 | 0.451 |
| sgp130 | 1.15 | 0.077 | 1.04 | 0.649 |
| sE-SELECTIN | 0.97 | 0.822 | 0.95 | 0.674 |
| Leptin | 0.59 | 0.075 | 0.91 | 0.681 |
| sEGFR | 1.24 | 0.077 | 1.05 | 0.694 |
| MMP-2 | 1.12 | 0.316 | 1.04 | 0.714 |
| PTH | 1.23 | 0.396 | 1.09 | 0.737 |
| MMP-9 | 1.06 | 0.769 | 0.95 | 0.786 |
| TPO | 1.13 | 0.635 | 1.04 | 0.860 |
| sIL-6R | 1.07 | 0.387 | 1.01 | 0.864 |
| sIL-1RII | 1.16 | 0.261 | 1.02 | 0.874 |
| IGFBP-1 | 1.02 | 0.941 | 0.97 | 0.910 |
| tPAI-1 | 1.13 | 0.229 | 1.00 | 0.974 |
| MCP-1 | 1.09 | 0.470 | 1.00 | 0.982 |

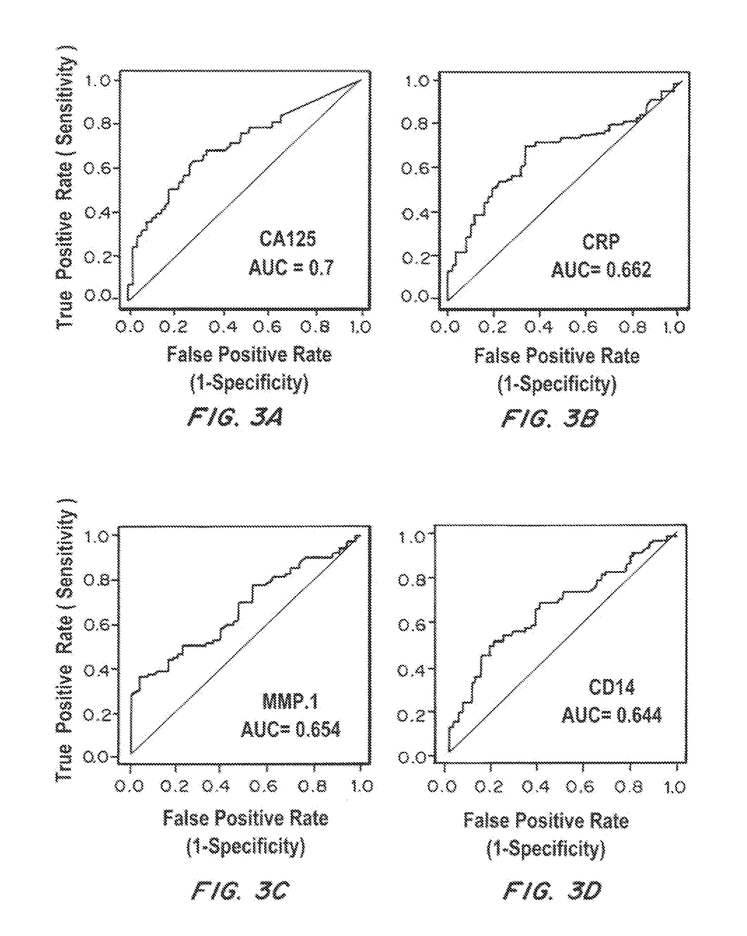

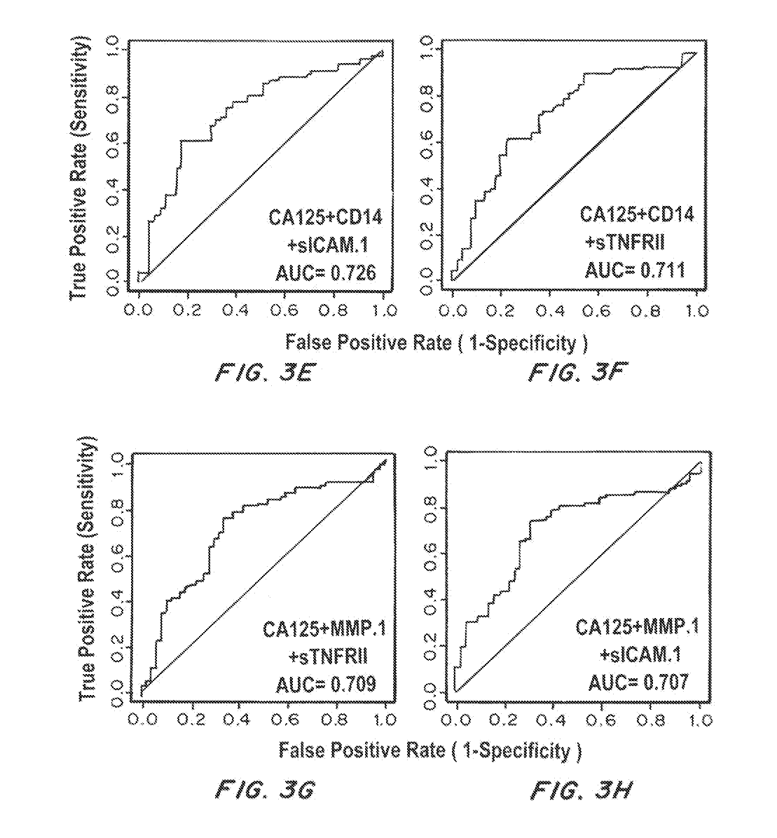

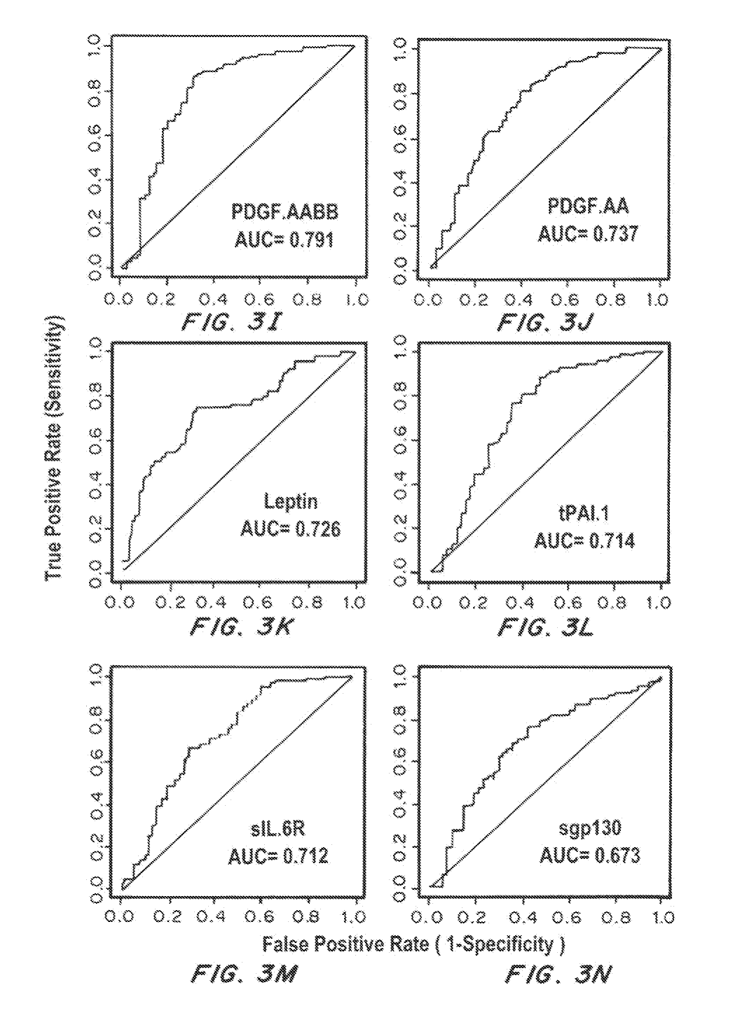

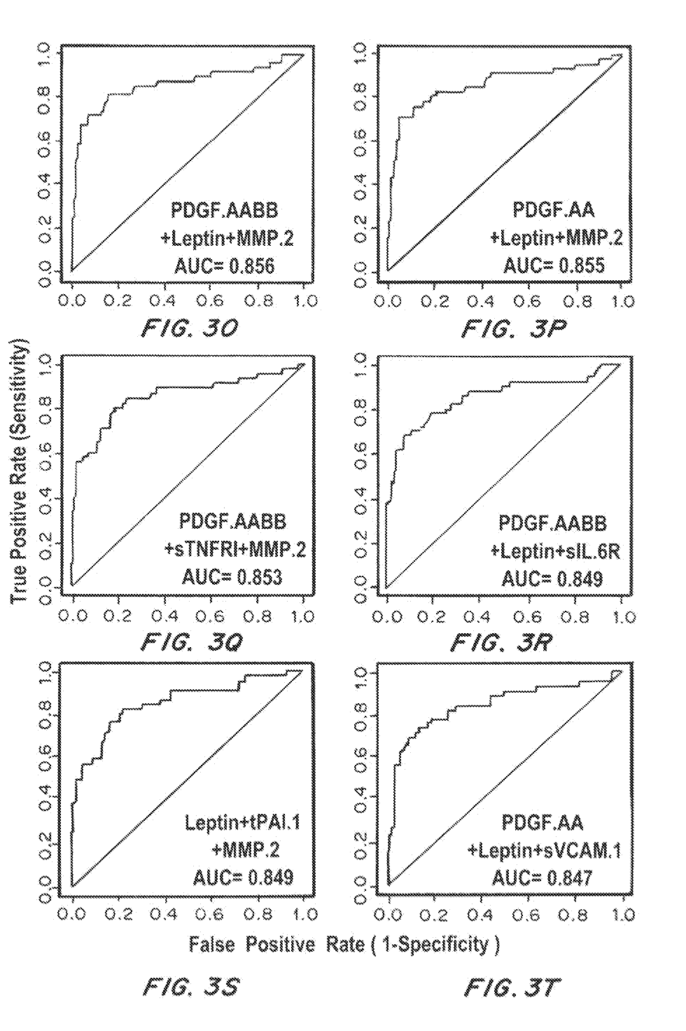

ROC analysis was also performed to identify individual molecules and 3-protein models that can best distinguish RM samples from cancer patients (PD+RC) or HC. The two best performing molecules that can distinguish RM from cancer are CA125 (AUC=0.7) and CRP (AUC=0.62) (FIGS. 3A-3D), while the best molecules which can separate RM and HC are PDGF-AAIBB, PDGF-AA and Leptin (AUC=0.79, 0.74 and 0.73, respectively, FIGS. 3I-3N). Combinations of proteins only slightly improved AUC (FIGS. 3E-3T).

### Example 4

### Serum Protein Profile at the PD Stage has Limited Prognostic Value

The impact of individual protein levels on survival was assessed using Kaplan-Meier analysis of 102 patients with survival data. The patients were assigned to the low or high expression groups based on the protein expression for each protein. As the best cutoff points were not known, eight cut-off points ranging from 30th percentile to 65th percentile of expression values (FIGS. 6A-6R) were systematically evaluated. After the patients were assigned to one or the other group, log rank test was used to determine survival differences between the two groups. Survival analyses were performed separately for the PD, RC and RM samples. Using PD samples, only five proteins showed marginally significant associations with survival (FIG. 4A). The prognostic value of multivariate models that contain 3, 4 or 5 proteins were then evaluated. For this purpose, k-means was used to cluster the patients into two groups based on the protein levels and Kaplan-Meier analyses were used to determine survival differences between the two clusters. Unfortunately, the multivariate models did not significantly improve the prognostic value of serum proteins measured at the PD stage (FIGS. 4B-4E).

### Example 5

### Five Serum Proteins at RM Stage Accurately Predict Therapeutic Outcomes

Eleven proteins (sICAM1, sTNFR-II, RANTES, sgp130, CA15-3, MIG, MMP-2, sVCAM-1, TPO, sTNFR-I and MDC) measured at the RM stage can individually predict overall survival of OC patients (FIG. 4A and FIGS. 6A-6AC). Among these proteins, five (sICAM1, sVCAM1, sgp130, MMP2, sTNFR-II) perform the best in separating the RM patients into two subgroups with distinct prognosis and sICAM-1 had the best prognostic value (HR=19.01, p=10⁻⁴, FIGS. 5A-5K). The prognostic value of all 5 models using 4 of the 5 proteins and the 5-protein model (FIGS. 5A-5K) was also evaluated. All five 4-protein models have excellent prognostic potential while the 5-protein model has the best performance (p=10⁻⁴ and MR=18.91). In the five-protein model, only one of the 29 patients in Cluster 1 did not survive, while 9 of the 16 patients in cluster 2 died during the follow-up period. Interestingly, the heat-map of protein expression (FIG. 5L) clearly shows that the patients with poor survival have higher expression levels for the five proteins.

### REFERENCES

1. Jemal A, Siege, R, Xu J, et al.: Cancer statistics. CA Cancer J Clin 60:277-300, 2010
2. Jemal A, Bray F, Center M M, et al.: Global cancer statistics. CA Cancer J Clin 61:69-90, 2011
3. Gagnon A, Ye B: Discovery and application of protein biomarkers for ovarian cancer. Curr Opin Obstet Gynecol 20:9-13, 2008
4. Hennessy B T, Coleman R L, Markman M: Ovarian cancer. Lancet 374:1371-1382, 2009
5. Langmar Z, Nemeth M, Vlesko G, et al.: HE4—a novel promising serum marker in the diagnosis of ovarian carcinoma. Eur J Gynaecol Oncol 32:605-610, 2011
6. Yurkovetsky Z, Skates S, Lomakin A, et al.: Development of a multimarker assay for early detection of ovarian cancer. J Clin Oncol 28:2159-2166, 2010
7. Clarke C H, Yip C, Badgwell D, et al.: Proteomic biomarkers apolipoprotein Al, truncated transthyretin and connective tissue activating protein III enhance
   the sensitivity of CA125 for detecting early stage epithelial ovarian cancer. Gynecol Oncol 122:548-553, 2011
8. Autelitano D J, Raineri L, Knight K, et al.: Performance of a multianalyte test as an aid for the diagnosis of ovarian cancer in symptomatic women. J Transl Med 10:45, 2012
9. Edgell T, Martin-Roussety G, Barker G, et al.: Phase II biomarker trial of a multimarker diagnostic for ovarian cancer. J Cancer Res Clin Oncol 136:1079-1088, 2010
10. Hefler-Frischmuth K, Hefler L A, Heinze G, et al.: Serum C-reactive protein in the differential diagnosis of ovarian masses. Eur J Obstet Gynecol Reprod Biol 147:65-68, 2009
11. Gorelik E, Landsittel D P, Marrangoni A M, et al.: Multiplexed immunobead-based cytokine profiling for early detection of ovarian cancer.

### Cancer Epidemiol

### Biomarkers Prey 14:981-987, 2005

12. Antovska S V, Bashevska N, Aleksioska N: Predictive values of the ultrasound parameters, CA-125 and risk of malignancy index in patients with ovarian cancer. Klin Onkol 24:435-442, 2011
13. Das P M, Bast R C, Jr.: Early detection of ovarian cancer. Biomark Med 2:291-303, 2008
14. Jelovac D, Armstrong D K: Recent progress in the diagnosis and treatment of ovarian cancer. CA Cancer J Clin 61:183-203, 2011
15. Grivennikov S I, Greten F R, Karin M: Immunity, inflammation, and cancer. Cell 140:883-899, 2010
16. Wu Y J Zhou B P: Inflammation: a driving force speeds cancer metastasis. Cell Cycle 8:3267-3273, 2009
17. Disis M L: Immune regulation of cancer. J Clin Oncol 28:4531-4538, 2010
18. Rustin G J, Timmers P, Nelstrop A, et al.: Comparison of CA-125 and standard definitions of progression of ovarian cancer in the intergroup trial of cisplatin and paclitaxel versus cisplatin and cyclophosphamide. J Clin Oncol 24:45-51, 2006
19. Kim K, Visintin I, Alvero A B, et al.: Development and validation of a protein-based signature for the detection of ovarian cancer. Clin Lab Med 29:47-55, 2009
20. Visintin I, Feng Z, Longton G, et al.: Diagnostic markers for early detection of ovarian cancer. Clin Cancer Res 14:1065-1072, 2008
21. Mor G, Visintin I, Lai Y, et al.: Serum protein markers for early detection of ovarian cancer. Proc Natl Acad Sci USA 102:7677-7682, 2005
22. Yip P, Chen T H, Seshaiah P, et al.: Comprehensive serum profiling for the discovery of epithelial ovarian cancer biomarkers. PLoS One 6:e29533, 2011
23. Hogdall C, Fung E T, Christensen I J, et al.: A novel proteomic biomarker panel as a diagnostic tool for patients with ovarian cancer. Gynecol Oncol 123:308-313, 2011
24. Zhu C S, Pinsky P F, Cramer D W, et al.: A framework for evaluating biomarkers for early detection: validation of biomarker panels for ovarian cancer. Cancer Prey Res (Phila) 4:375-383, 2011
25. Nolen B, Velikokhatnaya L, Marrangoni A, et al.: Serum biomarker panels for the discrimination of benign from malignant cases in patients with an adnexal mass. Gynecol Oncol 117:440-445, 2010
26. Amonkar S D, Bertenshaw G P, Chen T H, et al.: Development and preliminary evaluation of a multivariate index assay for ovarian cancer. PLoS One 4:e4599, 2009
27. Havrilesky L J, Whitehead C M, Rubatt J M, et al.: Evaluation of biomarker panels for early stage ovarian cancer detection and monitoring for disease recurrence. Gynecol Oncol 110:374-382, 2008
28. Zheng Y, Katsaros D, Shan S J, et al.: A multiparametric panel for ovarian cancer diagnosis, prognosis, and response to chemotherapy. Clin Cancer Res 13:6984-6992, 2007
29. Yurkovetsky Z, Ta'asan S, Skates S, et al.: Development of multimarker panel for early detection of endometrial cancer. High diagnostic power of prolactin. Gynecol Oncol 107:58-65, 2007
30. Toriola A T, Grankvist K, Agborsangaya C B, et al.: Changes in pre-diagnostic serum C-reactive protein concentrations and ovarian cancer risk: a longitudinal study. Ann Oncol 22:1916-1921, 2011
31. Lundin E, Dossus L, Clendenen T, et al.: C-reactive protein and ovarian cancer: aprospective study nested in three cohorts (Sweden, USA, Italy). Cancer Causes Control 20:1151-1159, 2009
32. Burger R A, Darcy K M, DiSaia P J, et al.: Association between serum levels of soluble tumor necrosis factor receptors/CA 125 and disease progression in patients with epithelial ovarian malignancy: a gynecologic oncology group study. Cancer 101:106-115, 2004
33. Baron-Hay S, Boyle F, Ferrier A, et al.: Elevated serum insulin-like growth factor binding protein-2 as a prognostic marker in patients with ovarian cancer. Clin Cancer Res 10:1796-1806, 2004
34. Lu D, Kuhn E, Bristow R E, et al.: Comparison of candidate serologic markers for type I and type II ovarian cancer. Gynecol Oncol 122:560-566, 2011
35. Ben-Hamo R, Efroni S: Biomarker robustness reveals the PDGF network as driving disease outcome in ovarian cancer patients in multiple studies. BMC Systems Biology 6:3, 2012
36. Robert A: Overview of anti-angiogenic agents in development for ovarian cancer. Gynecologic Oncology 121:230-238, 2011
37. Rahbari N, Schmidt T, Falk C, et al.: Expression and prognostic value of circulating angiogenic cytokines in pancreatic cancer. BMC Cancer 11:286, 2011
38. Steffensen K D, Waldstrorn M, Brandslund I, et al.: Prognostic impact of prechemotherapy serum levels of HER2, CA125, and HE4 in ovarian cancer patients. Int J Gynecol Cancer 21:1040-1047, 2011
39. Mury D, Woelber L, Jung S, et al.: Prognostic and predictive relevance of CA-125 at primary surgery of ovarian cancer. J Cancer Res Clin Oncol 137:1131-1137, 2011
40. Tang A, Kondalsamy-Chennakesavan S, Ngan H, et al.: Prognostic value of elevated preoperative serum CA125 in ovarian tumors of low malignant potential: A multinational collaborative study (ANZGOG0801). Gynecol Oncol, 2012
41. Lee C K, Friedlander M, Brown C, et al.: Early decline in cancer antigen 125 as a surrogate for progression-free survival in recurrent ovarian cancer. J Natl Cancer Inst 103:1338-1342, 2011
42. Skaznik-Wikiel M E, Sukumvanich P, Beriwal S, et al.: Possible use of CA-125 level normalization after the third chemotherapy cycle in deciding on chemotherapy regimen in patients with epithelial ovarian cancer: brief report. Int J Gynecol Cancer 21:1013-1017, 2011
43. van Altena A M, Kolwijck E, Spanjer M J, et al.: CA125 nadir concentration is an independent predictor of tumor recurrence in patients with ovarian cancer: a population-based study. Gynecoi Oncol 119:265-269, 2010

We claim:

1. A method for assessing therapeutic outcome of a treatment for ovarian cancer comprising determining the amount of one or more proteins in a blood sample from a ovarian cancer patient who is at remission after treatment, wherein the one or more proteins are selected from the group consisting of sICAM1, sTNFR-II, RANTES, sgp130, MMP-2, CA15-3, MIG, sVCAM-1, TPO, sTNFR-I and MDC and combinations thereof, and wherein elevated serum amounts of the one or more proteins or reduced level of MDC relative to a control indicates that the subject has poor overall survival relative to subjects in remission for ovarian cancer having lower serum amounts of the one or more serum proteins or higher MDC.

2. The method of claim 1, wherein the amounts of at least two of the one or more proteins are determined.

3. The method of claim 1, wherein the amounts of at least three of the one or more proteins are determined.

4. The method of claim 1, wherein the amounts of at least four of the one or more proteins are determined.

5. The method of claim 1, wherein the amounts of at least five of the one or more proteins are determined.

6. The method of claim 1, wherein the amounts of at least six of the one or more proteins are determined.

7. The method of claim 1, wherein the amounts of at least seven of the one or more proteins are determined.

8. The method of claim 1, wherein the amounts of at least eight of the one or more proteins are determined.

9. The method of claim 1, wherein the amounts of at least nine of the one or more proteins are determined.

10. The method of claim 1, wherein the amounts of at least ten of the one or more proteins are determined.

11. The method of claim 1, wherein the amounts of all eleven of the one or more proteins are determined.

12. The method of claim 1, furthering including the step of determining serum amounts of CA125 and or human epididymis protein 4 (HE4), wherein elevated serum amounts of CA125 and/or HE4 indicates that the subject has poor overall survival relative to subjects in remission for ovarian cancer having lower amounts of the one or more serum proteins.

13. A method for treating ovarian cancer comprising administering to a subject in need thereof one or more chemotherapeutic agents in an amount or for a duration effective to reduce serum levels of one or more proteins selected from the group consisting of sICAM, sVCAM1, sTNFR-II, sgp130, MMP2, CA15-3, MIG, sVCAM-1, TPO, sTNFR-I, and MDC and combinations thereof.

14. The method of claim 13, wherein the amounts of at least two of the one or more proteins are reduced.

15. The method of claim 13, wherein the amounts of at least three of the one or more proteins are reduced.

16. The method of claim 13, wherein the amounts of at least four of the one or more proteins are reduced.

17. The method of claim 13, wherein the amounts of five of the one or more proteins are reduced.

18. The method of claim 13, wherein the amounts of six of the one or more proteins are reduced.

19. The method of claim 13, wherein the amounts of seven of the one or more proteins are reduced.

20. A method for selecting a drug for the treatment of ovarian cancer comprising administering the drug to a non-human animal model of ovarian cancer, determining the amount of one or more proteins in a blood sample from the non-human animal model, wherein the one or more proteins are selected from the group consisting of sICAM, sVCAM1, sTNFR-II, sgp130, MMP2, and combinations thereof, and selecting the drug that reduces the amounts of the one or more proteins. (Covering non-human animal model is not very useful)
